SEQUENCE LISTING (10 sequences)

SEQ ID NO: 1 - protein, 214 aa, Artificial Sequence
  Synthetic- Light chain (1 and 2) of trastuzumab, a humanized anti-HER2 monoclonal antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, Artificial Sequence
  Synthetic- Heavy chain (1 and 2) of trastuzumab, a humanized anti-HER2 monoclonal antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, Artificial Sequence
  Synthetic- Light chain of pertuzumab, a humanized anti-HER2 monoclonal antibody
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 448 aa, Artificial Sequence
  Synthetic- Heavy chain of pertuzumab, a humanized anti-HER2 monoclonal antibody
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 330 aa, Homo sapiens [sequence omitted]

SEQ ID NO: 6 - DNA, 20 bp, Artificial Sequence
  Synthetic- primer for mouse Cd47
cccttgcatcgtccgtaatg

SEQ ID NO: 7 - DNA, 20 bp, Artificial Sequence
  Synthetic- primer for mouse Cd47
ggataagcgcgatgccatgg

SEQ ID NO: 8 - DNA, 20 bp, Artificial Sequence
  Synthetic- primer for human Cd47
atcgagctaaaatatcgtgt

SEQ ID NO: 9 - DNA, 20 bp, Artificial Sequence
  Synthetic- primer for human Cd47
ctactgaagtatacgtaaag

SEQ ID NO: 10 - DNA, 20 bp, Artificial Sequence
  Synthetic- primer for GFP control
gggcgaggagctgttcaccg

(19) United States
(12) Patent Application Publication
(10) Pub. No.: US 2022/0049015 A1
(43) Pub. Date: Feb. 17, 2022

(54) COMPOSITIONS AND METHODS FOR THE TREATMENT AND/OR PREVENTION OF HER2+ CANCERS

(71) Applicant: DUKE UNIVERSITY, Durham, NC (US)

(72) Inventors: Herbert Lyerly, Durham, NC (US); Zachary Hartman, Durham, NC (US)

(73) Assignee: DUKE UNIVERSITY, Durham, NC (US)

(21) Appl. No.: 17/297,821

(22) PCT Filed: Nov. 27, 2019
(86) PCT No.: PCT/US2019/063561

§ 371 Date: May 27, 2021

Related U.S. Application Data
(60) Provisional application No. 62/771,641, filed on Nov. 27, 2018.

(51) Int. Cl. C07K 16/32; A61K 39/395; C07K 16/28; C07K 14/705; A61P 35/00

(52) U.S. Cl. CPC C07K 16/32; A61K 39/3955; A61K 2039/507; C07K 14/70503; A61P 35/00; C07K 16/2803

(57) ABSTRACT

The present disclosure provides compositions and methods for the treatment of HER2⁺ cancers in a subject. The present disclosure provides a combination therapy of a HER2 antibody and a CD47 antagonist. The method activate an anti-tumor response that comprises activating the antibody dependent cellular phagocytosis (ADCP) within the subject.

### CROSS REFERENCE TO RELATED APPLICATIONS

This application claims priority to U.S. Provisional Application No. 62/771,641 filed on Nov. 27, 2018, the contents of which are incorporated by reference in its entirety.

### STATEMENT REGARDING FEDERAL FUNDING

This invention was made with government support under 5K12CA100639-09 and T32CA009111 from the National Institutes of Health and W81XWH-12-1-0574 from the Army Medical and Material Command (ARMY/MRMC). The government has certain rights in the invention.

### BACKGROUND

Approximately 20% of Breast Cancers (BC) overexpress HER2, which is recognized as an oncogenic driver of an aggressive cancer phenotype with a poor prognosis (1, 2). Monoclonal antibodies (mAbs) targeting HER2 were developed in the 1980s to inhibit HER2 oncogenic signaling, leading to the clinical development and regulatory approval of Trastuzumab in 1998 for metastatic HER2 overexpressed BC, followed by clinical trials of Trastuzumab for use in the adjuvant setting. Following its approval, additional HER2 targeting mAbs have been generated to improve outcomes (3, 4). However, the clinical benefit associated with HER2 mAb therapies in patients with HER2 overexpressing BC remains heterologous and metastatic HER2+ BC remains incurable (5, 6). Consequently, mechanistic studies of the antitumor mechanism(s) of action (MOA) of Trastuzumab and its resistance remain critical, not only to improve outcomes in patients with HER2+ BC, but also to gain insight into mechanisms that would extend mAb therapies to other types of cancers.

While suppression of HER2 signaling was a primary focus of early mechanistic studies, subsequent studies also focused on the role of immunity in mediating the antitumor effects of Trastuzumab (7). In particular, studies have shown the interaction of anti-HER2 antibodies with Fcγ-receptors (FCGR) expressed on innate immune cells such as macrophages, monocytes, natural killer (NK) cells and dendritic cells may be involved in its therapeutic activity (8, 9). The consequences of crosstalk with FCGR-bearing immune cells (8-10) are supported by the clinical observation that some host FCGR polymorphisms are associated with improved clinical outcome in HER2+ BC patients treated with Trastuzumab (11). Specifically, several studies have suggested the importance of these receptors in mediating Antibody-Dependent-Cellular-Cytotoxicity (ADCC), through NK cells or neutrophils for Trastuzumab efficacy (8, 9, 12-14). However, other studies have suggested the importance of adaptive immunity in mediating Trastuzumab efficacy, indicating that T cells may be critical for its antitumor MOA (8, 15).

While multiple MOAs involving either innate or adaptive immunity are possible, an underexplored mechanism is through mAb engagement of FCGRs to stimulate macrophage mediated Antibody- Dependent-Cellular-Phagocytosis (ADCP). Inconsistent reports about the role of ADCP exist, with a recent study demonstrating the ability of Trastuzumab to elicit ADCP (16), while another study suggests that Trastuzumab-mediated ADCP triggers macrophage immunosuppression in HER2+ BC (17). These disparate results may be partially attributed to the use of a wide range of tumor models (many not specifically driven by active HER2-signaling), as well as the use of different HER2-specific mAb clones of varied isotypes, which can elicit a range of different responses from various FCGRs (18, 19). Thus, the immunologic basis for the activity of Trastuzumab remains inconclusive, but could be effectively investigated through the development and use of appropriate HER2 targeting mAbs and model systems.

### SEQUENCE LISTING

A Sequence Listing accompanies this application and is submitted as an ASCII text file of the sequence listing named “2019-11-22_155554.00524_ST25.txt” which is 15.9 kb in size and was created on Nov. 22, 2019. The sequence listing is electronically submitted via EFS-Web with the application and is incorporated herein by reference in its entirety.

### BRIEF SUMMARY OF THE INVENTION

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect, the present disclosure provides a method for treating a HER2/neu positive cancer in a subject in need thereof. The method comprises administering to the subject a therapeutically effective amount of a HER2 antibody comprising an IgG Fc portion capable of binding Fcγ-receptor (FCGR) and activating the antibody dependent cellular phagocytosis (ADCP) and a CD47 antagonist such that the cancer is treated in the subject.

In another aspect, the present disclosure provides a pharmaceutical composition comprising at least one HER2 antibody comprising an IgG Fc portion capable of binding Fcγ-receptor (FCGR) and activating the antibody dependent cellular phagocytosis (ADCP) and a CD47 antagonist for the treatment of HER2/neu positive cancer.

In yet another aspect, the present disclosure provides a method comprising detecting in a tumor sample HER2/neu positive and CD47 positive tumor cells; and administering to the subject a therapeutically effective amount of a HER2 antibody comprising an IgG Fc portion capable of binding Fcγ-receptor (FCGR) and activating the antibody dependent cellular phagocytosis (ADCP) and a CD47 antagonist if both HER2⁺ and CD47⁺ tumor cells are detected.

Another aspect of the present disclosure provides all that is described and illustrated herein.

### BRIEF DESCRIPTION OF THE FIGURES

FIG. 1. Generation of murine Trastuzumab and studies revealing its dependence on Antibody-dependent-cellular-phagocytosis (ADCP) by tumor-associated macrophages (TAMs). (A) Cartoon presentation of Trastuzumab and 4D5 antibodies used in this study. (B) MM3MG cells expressing human HER2Δ16 were implanted into the mammary fat pads (1×10⁶ cells) of Balb/c mice. Trastuzumab (human IgG1) or 4D5 (mouse IgG2A) were administered weekly (200 μg per mice). n=8-10. (C) Tumors (>1000 mm³ volume) were processed into single cell suspensions, and TAMs (% CD11b+ F4/80+ LY6G− LY6C− of CD45+ cells) were analyzed by FACS. n=8-10. (D) Experiment as in FIG. 1B was repeated in SCID-Beige animals. n=8-10. (E) Experiment in SCID-Beige was repeated using neutrophils-depleting anti-LY6G antibodies (clone IA8, 300 μg per mice biweekly). (F-G) To deplete macrophages, SCID-Beige mice were pre-treated with anti-CSF1R antibody (clone AF S98, 300 μg, 3 times per week) for two weeks. (F) Macrophage depletion were verified by FACS. (G) 4D5-IgG2A injection were performed, with anti-CSF1R treatment maintained throughout the experiment. n=8. (H) Trastuzumab/4D5 induced ADCP of HER2+ BC cells by Bone-marrow-derived-macrophages (BMDM). MM3MG-HER2Δ16 cells were labeled with Brilliant Violet 450 Dye, and co-cultured with BMDM (3:1 ratio) with control or anti-HER2 antibodies (10 μg/mL). ADCP rates were measured by percentage of BMDM uptake of labeled tumor cells (CD45+and BV450+), and Antibody-dependent-cellular-cytotoxicity (ADCC) rates were measured by percentage of dying free tumor cells (CD45− and LIVE/DEAD stain+). ADCP inhibitor (Latrunculin A) or ADCC inhibitor (Concanamycin A) were added as assay controls. n=3, Experiment has been repeated three separate times. (B, D, E and G) Tumor growth were measured with caliper-based tumor measurement over time. Two-way ANOVA test with Tukey's multiple comparisons (C, F and H) One-way ANOVA test with Tukey' s multiple comparisons. All data represent mean ±SEM, **P<0.01, ***P<0.001, ****P<0.0001.

FIG. 2. The Antibody-dependent-cellular-phagocytosis (ADCP) activity of mouse Trastuzumab (4D5) requires the engagement with Fcγ-receptors (FCGR) and is IgG2A isotype dependent. (A) Fcγ-receptors are required for 4D5-induced ADCP of HER2+ BC cells by Bone-marrow-derived-macrophages (BMDM) in vitro. BMDM were generated from wild type and Fcer1g^{-/-} mice, and ADCP experiment were performed with the conditions described in FIG. 1E. (B-C) FCGR is required for the antitumor activity of 4D5 therapy. (B) Wild type or Fcer1g^{-/-} Balb/c mice were implanted with MM3MG-HER2Δ16 cells as before (FIG. 1B). 4D5-IgG2A or control antibodies were administered weekly (200 μg per mice intraperitoneally) and tumor growth were measured. n=5. (C) Tumor-associated macrophages (TAMs) from tumors in FIG. 2B were analyzed by FACS. n=4-5. (D-F) The ADCP activity of 4D5 is IgG2A isotype dependent. (D) MM3MG-HER2Δ16 tumor growth in mice were repeated using 4D5 antibodies containing the mouse IgG1 as comparison to previous IgG2A isotype. n=8-10. (E) ADCP experiments with BMDM cultures were performed using 4D5-IgG1 versus 4D5-IgG2A antibody isotypes. n=4. (F-H) Mouse FCGR signaling activation assay. MM3MG breast cancer cells expressing HER2 were plated and treated with indicated antibodies concentrations for 1 hour. Jurkat cells containing NFAT-luciferase reporter and expressing mouse FCGR1 (F), FCGR3 (G) or FCGR4 (H) were added to the target cells containing antibodies and co-cultured for 4 hours. FCGR signaling activation were assessed by luciferase activity quantification. n=4. (A, C, and E) One-way ANOVA with Tukey's multiple comparisons. (B, D, F, G and H) Two-way ANOVA test with Tukey's multiple comparisons to control IgG group. All data represent mean ±SEM, *P<0.05, ***P<0.001, ****P<0.0001.

FIG. 3. CD47 suppresses the anti-tumor activity of mouse Trastuzumab (4D5). (A) CD47 knockout cells were generated from MM3MG-HER2Δ16 cells using CRISPR-Cas9 technology. A control GFP knockout line was generated in parallel. Control and CD47-KO MM3MG-HER2Δ16 cells were labeled with Brilliant Violet 450 Dye, and incubated with Bone-marrow-derived-macrophages (BMDM) at 3:1 ratio with control or 4D5 antibodies (10 μg/mL). Antibody-dependent-cellular-phagocytosis (ADCP) and cytotoxicity (ADCC) activity were measured by as described in FIG. 1H. n=3. Experiment has been repeated two separate times using CD47-KO clones containing a different guide RNA. (B) Secreted cytokines and chemokines by macrophages from co-culture experiment with HER2+ BC were analyzed using the Luminex platform. Additional cytokines detected can be found in FIG. 13. n=3. (C-D) Control and CD47-KO MM3MG-HER2Δ16 cells were implanted into mouse mammary fat pads and treated with 4D5-IgG2A or control antibodies as described before. TAMs were analyzed by FACS after tumor volume reached >1000 mm³. n=5. (E-F) Cd47 overexpressing cells (CD47-OE) were generated in MM3MG-HER2416 cells after transduction with Cd47 cDNA under control of the EF1s promoter. CD47-OE tumor cell growth were compared to parental MM3MG-HER2Δ16 cells in mice treated with control antibody or 4D5-IgG2A. TAMs were analyzed by FACS. n=5. (A, B, D and F) One-way ANOVA with Tukey's multiple comparisons test. (C and E) Two-way ANOVA test with Tukey's multiple comparisons. All data represent mean±SEM, *P<0.05, ***P<0.001, ****P<0.0001.

FIG. 4. CD47 Blockade increased therapeutic efficacy of mouse Trastuzumab and augments tumor-associated macrophage (TAMs) expansion and phagocytosis. (A) Tumor growth experiment (as in FIG. 1B) were repeated using CD47 blockade antibody (MIAP410, 300 μg per mice) alone or in combination with 4D5-IgG2a. (B) TAM populations were analyzed by FACS after tumor volume reached >1000 mm³. Analysis of additional immune cell types are shown in FIG. 12D. Mean±SEM, n=8-10. (C) Repeat of similar tumor growth experiment and treatments in SCID-Beige mice. (D) TAM populations from SCID-Beige experiment were analyzed by FACS. n=10. (E) Schematic representation of in vivo Antibody-dependent-cellular-phagocytosis (ADCP) experiment. MM3MG-HER2Δ16 cells were labeled with Vybrant DiD dye and implanted (1×10⁶ cells) into mammary fat pads of Balb/c mice. Once tumor volume reaches ˜1000 mm³, mice were treated with either control antibody, 4D5-IgG2A (200 μg), or in combination with MIAP410 (300 μg). On the next day, tumors were harvested and tumor-phagocytic macrophages were quantified by FACS. (F) Representative FACS plots and graphical summary showing frequency of macrophages (CD11b+, F4/80+, LY6G−, LY6C−) that have phagocytosed DiD-labeled tumor cells. n=6. (G) Similar in vivo ADCP experiment were repeated in Fcer1g^{-/-} mice. n=8. (A and C) Two-way ANOVA test with Tukey's multiple comparisons. (B, D, G and G) One-way ANOVA test with Tukey's multiple comparisons. All data represent mean±SEM *P<0.05, **P<0.01, ***P<0.001, ****P<0.0001.

FIG. 5. CD47 blockade synergizes with mouse Trastuzumab therapeutic activity in a transgenic human HER2+ breast cancer (BC) mouse model. (A) Schematic representation of experiment using the endogenous human HER2 transgenic mouse model. Spontaneous breast tumors in the transgenic animals were induced with doxycycline diet. Four treatment arms were set up: Control IgG (200 μg weekly, n=15), CD47 blockade (MIAP410, 300 μg weekly, n=14), 4D5-IgG2A (200 μg weekly, n=16) and 4D5-IgG2A combined with MIAP410 (n=16). Individual animals were consecutively enrolled into a specific treatment arm as soon as palpable breast tumors were detected (˜200 mm³). (B) Survival of mice in each treatment arm, time of start is on the day of palpable tumor detection and treatment enrollment. Log-rank (Mantel-Cox) test for survival analysis, ****P<0.0001 of treatment vs control group, ##P<0.01 significant difference observed between “4D5” group vs “4D5+αCD47” group. (C) Tumor burden in animals from each treatment arm were measured over time after enrollment in treatment arm. Each individual animal develops 1 to 4 total tumors in their mammary fat pads. The total tumor burden per mice is shown. Animals were terminated when their total tumor volume reached >2000 mm³. (D) Tumors in the transgenic mice were harvested, processed into single cell suspensions, and analyzed by FACS. Each individual tumor were treated as an individual measurement. Mean±SEM, Control IgG n=23, αCD47 n=27, 4D5 n=38, 4D5+αCD47 n=32, One-way ANOVA with Tukey's multiple comparisons test, *P<0.05, **P<0.01, ***P<0.001.

FIG. 6. Single-cell transcriptome analysis of immune clusters within HER2+ BC after Trastuzumab with CD47 blockade therapy. HER2+ tumors from HER2Δ16 transgenic animals were isolated for Single-Cell RNA-Sequencing using 10× Genomics platform. Data from all tumors were pooled for clustering and gene expression analysis. (A) tSNE plots showing distinct clusters of immune cells in tumors from four treatment groups: control IgG, αCD47, 4D5-IgG2A or combination. (B-C) Heat map of relevant gene markers confirmed the various immune cell clusters in control tumors (B), and the expansion of macrophage clusters in the combination therapy treated tumors (C). Macrophages that contains tumor specific transcripts (e.g. hERBB2, Epcam, Krt8) were labeled as tumor phagocytic macrophages (Phag MΦ, predominantly found in combination treatment group).

FIG. 7. Differential gene expression analysis of TAM clusters in HER2+ BC after Trastuzumab with CD47 blockade therapy. (A-B) Differential gene expression analysis of gene signatures for IFN, pro-inflammation, chemotaxis and TLR/MyD88/NFkb pathways in M1-like MΦ clusters (A) and M2-like MΦ clusters (B) revealed how they were affected by the treatment regimens. (C) Differential gene expression analysis of immuno-regulatory gene signatures (wound-healing, ECM remodeling, growth factors, anti-inflammation) versus immuno-stimulatory gene signatures (pro-inflammation, chemotaxis, antigen presentation, phagocytosis/opsonization) among the three distinct macrophage clusters in the combined dataset.

FIG. 8. Human CD47 gene expression is a prognostic factor in HER2+ breast cancer and limits the therapeutic activity of Trastuzumab. (A-B) Kaplan-Meier survival curve for breast cancer (BC) patients METABRIC Dataset. (A) Stratified into low and high groups based on average expression of CD47 in all patients. (B) The same patient stratification based on disease subtype (ER+, HER2+ and TNBC). (C) CD47 knockout in human HER2+ BC line KPL-4 was generated using CRISPR-Cas9 approach. Control and CD47-KO KPL-4 cells were labeled with Brilliant Violet 450 Dye, and incubated with human monocytes-derived-macrophages (hMDM) at a 3:1 ratio, in the presence of control or Trastuzumab (10 μg/mL). Antibody-dependent-cellular-phagocytosis (ADCP) activity were measured by percentage of hMDM uptake of labeled KPL-4 cells (CD45+ and BV450+). Mean±SEM, biological replicates n=4. Experiment has been repeated using hMDMs generated from three healthy PBMC donors. (D) Control or CD47-KO KPL-4 cells were implanted into mammary fat pads of SCID-Beige Balb/c mice (5×10⁵ cells). Trastuzumab (50 μg) or control human IgG1 were administered weekly and tumor volume were measured. Two-way ANOVA test with Tukey's multiple comparisons, ****P<0.0001. (E) Tumor infiltrating macrophages (F4/80+ Gr1− CD11b+) populations were analyzed by FACS, except for “CD47-KO+Trastuzumab” group as no tumor growths have occurred. Mean±SEM, n=7. (F) Tumor-associated macrophages from control treated and trastuzumab treated tumors were sorted by FACS (F4/80+Gr1− CD11b+CD45+) and analyzed with RT-qPCR for the expression of pro- and anti-inflammatory genes. Mean±SEM, n=7. Multiple two-sided t-test. (C and E) One-way ANOVA test with Tukey's multiple comparisons, *P<0.05, **P<0.01, ***P<0.001.

FIG. 9. (A) Cell-based ELISA assay to determine 4D5 and Trastuzumab binding efficiency to human HER2 expressed on NMUMG cell lines. EC50 for each binding assay were calculated using non-linear regression curve fit, Assymetric Sigmoidal model in Graphpad Prism software. (B) Immune responses against Trastuzumab (a human antibody) in mice were assessed in Trastuzumab-treated mice (I.P. injection 200 μg) after 2 weeks post injection. ELISA assays using Trastuzumab as antigen were performed to determine anti-Trastuzumab responses in mouse serum. (C-D) HER2 signaling assays were performed using 293T cells stably transduced with dox-inducible HER2Δ16. Cells were treated with dox and transfected with luciferase reporter constructs for (C) MAPK/ERK or (D) AP-1/c-JUN pathways activation. 4D5 and Trastuzumab were added at titrated concentrations to inhibit HER2 signaling. The HER2-Tyrosine kinase inhibitor Lapatinib were used as positive assay control at the highest possible dose (500 nM) without inducing cell toxicity. (E) Trastuzumab effect on human HER2+ breast cancer growth (KPL4 and SKBR3 cells) in vitro were assessed by MTT assays 3 days post Trastuzumab treatment.

FIG. 10. (A) Tumors in FIG. 1A were harvested, processed into single cell suspensions, and tumor infiltrating immune cell populations (NK cells, CD4+ T cells and CD8+ T cells) were analyzed by FACS. (B-C) Anti-tumor specific T cell responses as measured by IFNγ ELISPOT against human HER2 peptides using mouse splenocytes from (B) MM3MG-HER2Δ16 orthotopic model or (C) HER2 transgenic model (described in FIG. 5A). (D) In vitro NK cell mediated ADCC assay were performed using NK.92 expressing mouse FCGR3 as effector cells and CEM.NKR expressing HER2 and luciferase as target cells. Results showed both Trastuzumab and 4D5 treatment enhanced NK-mediated ADCC in vitro. Mean±SEM, biological replicates n=4, two-sided t-test, ***P<0.001. (E) In vitro Complement-dependent-cytotoxicity (CDC) assay were performed using 25% human serum treatment (4 hours) on MM3MG-HER2Δ16 lines expressing luciferase. Results showed neither Trastuzumab or 4D5-IgG2A mAbs could enhance complement-mediated tumor cell killing. Mean±SEM, biological replicates n=4, One-Way ANOVA with Tukey's multiple comparisons.

FIG. 11. Clodronate Liposomes injections were used to deplete macrophages in SCID-beige mice before implantation of HER2+ MM3MG tumor (100 μL/mice, 2×/week). (A-B) Macrophages in spleen (A) and tumor (B) were analyzed by FACS. Mean±SEM, n=5, One-way ANOVA test, ***P<0.001. (C) Tumor growth were measured over time. Mean±SEM, n=5, Two-way ANOVA test with Tukey's multiple comparisons, ***P<0.001. (D-E) Anti-Ly6G antibody were used to deplete neutrophils (biweekly I.P, 300 μg/mice). FACS analysis showing neutrophils in spleen (D) and in tumor (E).

FIG. 12. Flow cytometry confirmations of (A) CD47 knock-out in MM3MG-HER2-Δ16. (B) CD47 overexpression in MM3MG-HER2-Δ16. (C) CD47 knock-out in KPL4. (D) mouse FCGR1 expression in Jurkat-NFAT-LUC. (E) mouse FCGR3 expression in Jurkat-NFAT-LUC. (F) mouse FCGR4 expression in Jurkat-NFAT-LUC.

FIG. 13. Secreted cytokines and chemokines by macrophages from co-culture experiment with HER2+ BC and antibodies were analyzed using the Luminex platform. Supplementary to FIG. 3B

FIG. 14. (A) Additional FACS analysis of immune cell populations in the orthotopic HER2+ tumors from experiment shown in FIG. 4A. Mean±SEM, n=8-10. (B) Additional FACS analysis of immune cell populations in the HER2 transgenic tumors from experiment shown in FIG. 5. Mean±SEM, Control IgG n=23, aCD47 n=27, 4D5 n=38, 4D5+ αCD47 n=32. (A and B) One-way ANOVA test with Tukey's multiple comparisons, *P<0.05, **P<0.01, ***P<0.001.

FIG. 15. (A) Immunohistochemistry staining of CD68 of paraffin-embedded tumor samples derived from therapy experiments described in FIG. 4A. Representative images of tumors from each treatment groups are shown. Original magnification=20×. (B) Summary of CD68+ staining quantifications. n=30. One-way ANOVA test with Tukey's multiple comparisons. All data represent mean ±SEM, **P<0.01, ***P<0.001, ****P<0.0001.

FIG. 16. Table S1 Single-Cell RNA-seq analysis of total CD8+ T cell frequency in tumor and percentage of CD8+ T cells expressing cytotoxic markers (Ifng and Gzmb). Data shows the mean of replicates in each treatment group.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. For example, “about” may be about +/−10% of the numerical value.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject comprises a human. In other embodiments, the subject comprises a human suffering from a HER2-positive cancer. In certain embodiments, the subject comprises a human suffering from a HER2-positive breast cancer.

“Administration” as it applies to a human, primate, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent/compound, therapeutic agent/compound, diagnostic agent/compound, compound or composition to the subject, cell, tissue, organ, or biological fluid, and the like. “Administration” can refer, e.g., to therapeutic, pharmacokinetic, diagnostic, research, placebo, and experimental methods.

As is known in the art, a cancer is generally considered as uncontrolled cell growth. The methods of the present disclosure can be used to treat any cancer, and any metastases thereof, that expresses HER2/neu. Examples include, but are not limited to, breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma. In certain embodiments, the HER2-positive cancer comprises breast cancer.

Unless otherwise defined, all technical terms used herein have the same meaning as commonly understood by one of ordinary skill in the art to which this disclosure belongs.

The present disclosure provides a method for treating a HER2/neu positive cancer in a subject in need thereof, the method comprising, consisting of, or consisting essentially of administering to the subject a therapeutically effective amount of a HER2 antibody comprising an IgG Fc portion capable of binding Fcγ-receptor (FCGR) and activating the antibody dependent cellular phagocytosis (ADCP) and a CD47 antagonist such that the cancer is treated in the subject.

The inventors have found antitumor activity of HER2 antibodies with high A/I ratios was dependent on Fcγ-Receptor stimulation of tumor-associated-macrophages (TAM) and Antibody-Dependent-Cellular-Phagocytosis (ADCP). HER2 antibodies stimulated TAM activation and expansion, but did not require adaptive immunity, natural killer cells, and/or neutrophils. Moreover, inhibition of the innate immune ADCP checkpoint, CD47, significantly enhanced HER2-antibodiy mediated ADCP, TAM expansion and activation, resulting in the emergence of a unique hyper-phagocytic macrophage population, improved antitumor responses and prolonged survival. The present disclosure provides methods of treating HER2/neu positive cancers by administering a HER2 antibody isotype with a high A/I ratio (e.g., human IgG1) and an antagonist of CD47 in an amount in combination that is effective to treat the cancer.

Suitable HER2 antibodies for use in the present disclosure are any HER2 antibodies that can bind HER2 and have a proper isotype, i.e., isotypes of high activating-to-inhibitory ratio (A/I ratio), e.g., IgG Fc portion), capable of binding Fcγ-receptor (FCGR) and activating the antibody dependent cellular phagocytosis (ADCP), tumor-associated macrophages (TAM) or both. Suitable HER2 antibodies contain IgG Fc include HER2 antibodies that have a human IgG1 Fc portion. Suitable isotypes or Fc portions are isotypes with a high activating FcγR binding to inhibitory FcγR binding (A/I ratio, calculated by dividing the affinity of a specific IgG isotype for an activating receptor by the affinity for the inhibitory receptor). The term “high A/I ratio” as used herein refers to an A/I ratio of greater than 1.

Suitable HER2 antibodies are commercially available and known in the art. For example, suitable HER2 antibodies include, but are not limited to, for example, trastuzumab (Herceptin®; Genentech, South San Francisco, Calif.; SEQ ID NOs: 1-2), trastuzumab-dkst (trastuzumab biosimilar, also known as MYL-1401O; Ogivri™; Mylan Pharmaceuticals, Canonsburg, Pa.), ado-trastuzumab emtansine (trastuzumab covalently linked to the cytotoxic agent DM1; KADCYLA®, Genentech, South San Francisco, Calif.), pertuzumab (Perjeta®, Genentech, South San Francisco, Calif.; SEQ ID NOs: 3-4) and combinations thereof. One skilled in the art would also be able to modify HER2 antibodies that may not have the ideal Fc portion to contain a suitable Fc portion that is able to activate ADCP and TAM, for example, by swapping in the human IgG1 Fc portion into the antibody. In an exemplary embodiment, the HER2 antibody is trastuzumab.

It is contemplated that other HER2 antibodies can be engineered to be proper isotypes (e.g., high A/I ratio) capable of binding FCGR and activating ADCP and TAM within a subject. One skilled in the art would be able to select and engineer proper HER2 antibodies as described herein. Suitable IgGs include, but are not limited to, human IgG1 (e.g., UniProtKB-P01857 (SEQ ID NO: 5) or a sequence having at least 90% similarity to, preferably 95% similarity to the human IgG1 sequence and is capable of activating ADCP and TAM by binding FCGR. In some examples, the Fc portion is from human IgG1 or a polypeptide sequence having at least 90%, 91%, 92%, 93%, 94%, 95%, 96%, 97%, 98%, 99%, or 100% sequence identity to human IgG1.

Regarding the polypeptides disclosed herein, the phrases “% sequence identity,” “percent identity,” or “% identity” refer to the percentage of residue matches between at least two amino acid sequences aligned using a standardized algorithm. Methods of amino acid sequence alignment are well-known in the art. A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that may be used to align a known amino acid sequence with other amino acids sequences from a variety of databases.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Suitable CD47 antagonists are known in the art, and including CD47 inhibitors or CD47 antagonists that block the interaction and signaling of CD47 through signal-regulatory protein alpha (SIRPα), an inhibitory transmembrane receptor present on myeloid cells. Suitable CD47 antagonists, including CD47 inhibitors, are known in the art and commercially available, and include, but are not limited to, for example, MIAP301 (available from ThermoFisher Scientific, Waltham, Mass.; Santa Cruz Biotechnology, Dallas, Tex.; Novus Biologicals, Centennial, Colo.), MIAP410 (available from VWR, Radnor, Pa.; Bio X Cell, West Lebanon, N.H.), TTI-621 (described in US Patent Application No. 20180312563, incorporated by reference herein; Trillium Therapeutics Inc., Mississauga, Canada), CV1 (described in Weiskopf et al. (2013) Science 341(6141): 88-91, incorporated by reference herein), Hu5F9-G4 (described in Liu et al. (2015) PLoS One 10(9):e0137345, incorporated by reference herein), CC-90002 (Celgene, Summit, N.J.), B6H12 (available from ThermoFisher Scientific, Waltham, Mass.; Santa Cruz Biotechnology, Dallas, Tex.; Abcam, Cambridge, United Kingdom), 2D3 (available from ThermoFisher Scientific, Waltham, Mass.; Novus Biologicals, Centennial, Colo.) and combinations thereof. In an exemplary embodiment, the CD47 antagonist is MIAP410.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. The term “treating” can be characterized by one or more of the following: (a) the reducing, slowing or inhibiting the growth or proliferation of cancer cells or tumor cells (e.g., cancers or tumors), including reducing, slowing or inhibiting the growth or proliferation of HER2/neu⁺cancer cells; (b) preventing the further growth or proliferation of cancer cells, for example, breast cancer cells; (c) reducing or preventing the metastasis of cancer cells within a patient, (d) killing or inducing apoptosis of cancer cells, and (d) reducing or ameliorating at least one symptom of cancer. In one embodiment, the term treating is characterized by a reduction in the number of cancer cells in the subject, for example, reduction in the number of HER/neu⁺ cell, for example HER2⁺ breast cancer cells.

As used herein, the terms “effective treatment” refers to the treatment producing a beneficial effect, e.g., yield a desired therapeutic response without undue adverse side effects such as toxicity, irritation, or allergic response. A beneficial effect can take the form of an improvement over baseline, i.e., an improvement over a measurement or observation made prior to initiation of therapy according to the method. A beneficial effect can also take the form of reducing, inhibiting or preventing further growth of cancer cells, reducing, inhibiting or preventing metastasis of the cancer cells or invasiveness of the cancer cells or metastasis or reducing, alleviating, ameliorating, inhibiting or preventing one or more symptoms of the cancer or metastasis thereof. Such effective treatment may, e.g., reduce patient pain, reduce the size or number of cancer cells, may reduce or prevent metastasis of a cancer cell, or may slow cancer or metastatic cell growth.

The term “effective amount” or “therapeutically effective amount” refers to an amount sufficient to effect beneficial or desirable biological and/or clinical results. That result can be reducing, inhibiting or preventing the growth of cancer cells, reducing, inhibiting or preventing metastasis of the cancer cells or invasiveness of the cancer cells or metastasis, or reducing, alleviating, inhibiting or preventing one or more symptoms of the cancer or metastasis thereof, or any other desired alteration of a biological system. Effective amounts of the antagonists and antibody can be determined by a physician with consideration of individual differences in age, weight, tumor size, extent of infection or metastasis, and condition of the patient/subject. In some embodiments, the optimum effective amounts can be readily determined by one of ordinary skill in the art using routine experimentation.

As used herein, the terms “administering” and “administration” refer to any method of providing the treatment to the patient, for example, any method of providing a pharmaceutical composition to a subject. Such methods are well known to those skilled in the art and include, but are not limited to, oral administration, transdermal administration, administration by parenteral administration, including injectable such as intravenous administration, intra-arterial administration, intramuscular administration, intradermal administration, intrathecal administration and subcutaneous administration, rectal administration, sublingual administration, buccal administration, among others.

Administration can be continuous or intermittent. In various aspects, a preparation or combination of compounds can be administered therapeutically; that is, administered to treat an existing cancer.

In some embodiments, the CD47 antagonist is administered prior to the HER2 antibody. In other embodiments, the CD47 antagonist is administered co-currently with the HER2 antibody. Not to be bound by any theory, but it is thought that by inhibiting CD47 before or concurrently with administration of the HER2 antibody (or within a time frame in which the HER2 antibody is active within the subject) allows for the ability to block the downstream effects of CD47 signaling, allowing for increase in ADCP and increase TAM within the subject, increasing the efficacy of the HER2 antibody in being able to reduce the number cancer cells or inhibit further cancer growth within the subject.

In some embodiments, the subject comprises a human suffering from a HER2-positive cancer. In certain embodiments, the subject comprises a human suffering from a HER2-positive breast cancer.

The present disclosure also provides a method of detecting a subpopulation of patients in which the combination of HER2 antibody and CD47 antagonist would have an anti-tumor effect. This method includes screening of patients by detecting the presence of both a HER/neu+ positive cancer and the cancer expresses increased amounts of CD47 (CD47⁺) as compared to a control. As described in the examples, when CD47⁺ was present with BERT' cancer, the cancers were more resistant to anti-HER2 antibody therapy. In detecting cancers in which CD47 is elevated in the HER2⁺ cancer population, the present methods of treatment can be used to increase the efficacy of the HER2 antibody and increase the length of survival. Methods of detecting CD47⁺ cells are known in the art, and include, but are not limited to, detecting protein expression level on the surface (e.g., FACS, ELISA, Western Blot, etc.) or mRNA levels within the cells (e.g., RT-PCR, microarray analysis, northern blot analysis, in situ hybridization, etc.).

In one embodiment, the method further comprises detecting a HER2/neu⁺ CD47⁺ cancer within a subject before administering a HER2 antibody and a CD47 antagonist.

Pharmaceutical compositions comprising at least one HER2 antibody comprising an IgG Fc portion capable of binding Fcγ-receptor (FCGR) and activating the antibody dependent cellular phagocytosis (ADCP) and at least one CD47 antagonist are contemplated for the treatment of HER2/neu positive cancer. Any suitable HER2 antibody described herein is suitable for the pharmaceutical compositions. In a preferred embodiment, the HER2 antibody is trastuzumab, however, any HER2 antibody having a high A/I ratio is contemplated for use in the present compositions and methods.

In another embodiment, a method of comprising: detecting in a tumor sample HER2/neu positive and CD47 positive tumor cells; and administering to the subject a therapeutically effective amount of a HER2 antibody comprising an IgG Fc portion capable of binding Fcγ-receptor (FCGR) and activating the antibody dependent cellular phagocytosis (ADCP) and a CD47 antagonist in a subject in which both HER2⁺ and CD47⁺ tumor cells are detected. Patients that have HER2⁺CD47⁺ tumors may have the most efficacy with the use of the combination described herein.

The antibody and antagonist provided herein can be administered to a subject either alone, or in combination with a pharmaceutically acceptable excipient, in an amount sufficient to induce an appropriate anti-cancer response. It can generally be stated that a pharmaceutical composition comprising the compounds described herein may be administered at a dosage of 1 to 10 mgs/kg body weight, preferably 2 to 8 mgs/kg body weight, including all integer values within those ranges. The compounds may also be administered multiple times at these, or other, dosages. The compounds can be administered by using any techniques that are commonly known in cancer therapy. The optimal dosage and treatment regime for a particular patient can readily be determined by one skilled in the art of medicine by monitoring the patient for signs of disease and adjusting the treatment accordingly.

An effective amount of the compounds described herein may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of the compounds. Where there is more than one administration in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The present disclosure is not limited to dosing intervals that are spaced equally in time, but encompass doses at non-equal intervals, such as a priming schedule consisting of administration at 1 day, 4 days, 7 days, and 25 days, just to provide a non-limiting example.

A “pharmaceutically acceptable excipient”, “diagnostically acceptable excipient” or “pharmaceutically acceptable carrier” are used interchangeably and includes but is not limited to, sterile distilled water, saline, phosphate buffered solutions, amino acid-based buffers, or bicarbonate buffered solutions. An excipient selected and the amount of excipient used will depend upon the mode of administration. The pharmaceutically acceptable excipient or carrier are any that are compatible with the other ingredients of the formulation and not deleterious to the recipient. Pharmaceutically acceptable carrier can be selected on the basis of the selected route of administration and standard pharmaceutical practice for the compounds. The active agent may be formulated into dosage forms according to standard practices in the field of pharmaceutical preparations. See Alphonso Gennaro, ed., Remington's Pharmaceutical Sciences, 18th Ed., (1990) Mack Publishing Co., Easton, Pa. Suitable dosage forms may comprise, for example, tablets, capsules, solutions, parenteral solutions, injectable solutions, troches, suppositories, or suspensions. Administration may comprise an injection, infusion, oral administration, or a combination thereof. Formulations of the compounds or any other additional therapeutic agent(s) may be prepared for storage by mixing with physiologically acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

An effective amount for a particular subject/patient may vary depending on factors such as the condition being treated, the overall health of the patient, the route and dose of administration and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK). A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

The compounds according to the present disclosure may also be administered with one or more additional therapeutic agents or therapies, including, but not limited to, other chemotherapeutic agents, radiation, surgery, and the like. In one example, the compounds (e.g., HER2 antibody and CD47 antagonists) may be administered in combination with an additional HER2 antagonist. Suitable HER2 antagonists are known in the art and commercially available and include, but are not limited to, for example, lapatinib (TYKERB®, GlaxoSmithKline, Brentford, United Kingdom), neratinib (NERLYNX®, Puma Biotechnology, Los Angeles, Calif.), among others. Methods for co-administration with an additional therapeutic agents/therapies are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice:A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.).

Co-administration need not to refer to administration at the same time in an individual, but rather may include administrations that are spaced by hours or even days, weeks, or longer, as long as the administration of the compounds (and any other multiple therapeutic agents/therapies) is the result of a single treatment plan. By way of example, the first compound (HER2/neu antibody) may be administered prior to the second compound (CD47 antagonist), or the first compound may be administered concurrently with the second compound, or the first compound is administered after the second compound. This is not meant to be a limiting list of possible administration protocols.

An effective amount of a compound or any additional therapeutic agents/therapies or combinations thereof is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

The present disclosure also provides methods of enhancing the anti-tumor effect of a HER2 antibody by administering a CD47 antagonist to the subject in combination with the HER2 antibody. The CD47 antagonist is able to increase the ADCP and TAM (tumor-associated macrophages) within the tumor microenvironment, increasing the anti-tumor response to the cancer.

Yet another aspect of the present disclosure provides all that is disclosed and illustrated herein.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

### Her2 Mab in Combination with CD47/SIRP1α Inhibition

In this Example, the inventors developed and utilized fully murinized Trastuzumab mAbs (clone 4D5) with isotypes of different activating-to-inhibitory ratio (A/I ratio, calculated by dividing the affinity of a specific IgG isotype for an activating receptor by the affinity for the inhibitory receptor) (19), as well as clinical-grade Trastuzumab, to determine the MOA for Trastuzumab antitumor efficacy. These mAbs were tested in multiple settings to interrogate ADCC and ADCP, as well as the impact on HER2 signaling and complement-dependent cytotoxicity (CDC). To determine the antitumor efficacy of these HER2 mAbs, we employed orthotopic implantation of HER2+ murine BC cells (transformed using a constitutively active isoform of human HER2) in immunocompetent models, as well as Fcgr^{-/-}, immune-deficient backgrounds, and human HER2+ BC xenograft models. In addition, we utilized a novel transgenic HER2+ BC model driven by an oncogenic isoform of human HER2 to simulate an endogenous mammary tumor immune microenvironment (20, 21). Collectively, these studies revealed an essential role for tumor-associated macrophages (TAMs) in mediating the therapeutic activity of Trastuzumab through promoting ADCP of HER2+ tumor cells without evidence for significant induction of adaptive T cell responses against HER2. We also observed that this effect was subverted by innate mechanisms of immunosuppression in the tumor microenvironment that limit macrophage ADCP.

Previous studies have demonstrated that ADCP is principally regulated by anti-phagocytic “don't eat me” signals that are amplified in many cancers (22, 23). Chief among these is CD47, which has been shown to be highly expressed in different cancers and functions to suppress phagocytosis through binding to and triggering signaling of macrophage SIRPα (23, 24). Notably, CD47 expression is also upregulated in BC (25). As a potential means to subvert innate immune regulation and enhance ADCP and possibly alter the macrophage phenotype in HER2+ BC, we also targeted the CD47-SIRPα innate immune checkpoint. In this study, we demonstrate that TAM ADCP can be significantly enhanced by blocking the CD47-SIRPα checkpoint to enable Trastuzumab-mediated macrophage phagocytosis of HER2+ tumor cells. Collectively, these findings support the importance of the ADCP MOA, as well as suggest the therapeutic potential of utilizing CD47-SIRPα checkpoint blockade in combination with Trastuzumab in HER2+ BC and potentially in other resistant HER2+ cancers (i.e. gastric, bladder, etc.) (26).

### Generation of Murine Trastuzumab (4D5) and its Antitumor Dependence on ADCP by Tumor-Associated Macrophages (TAMs)

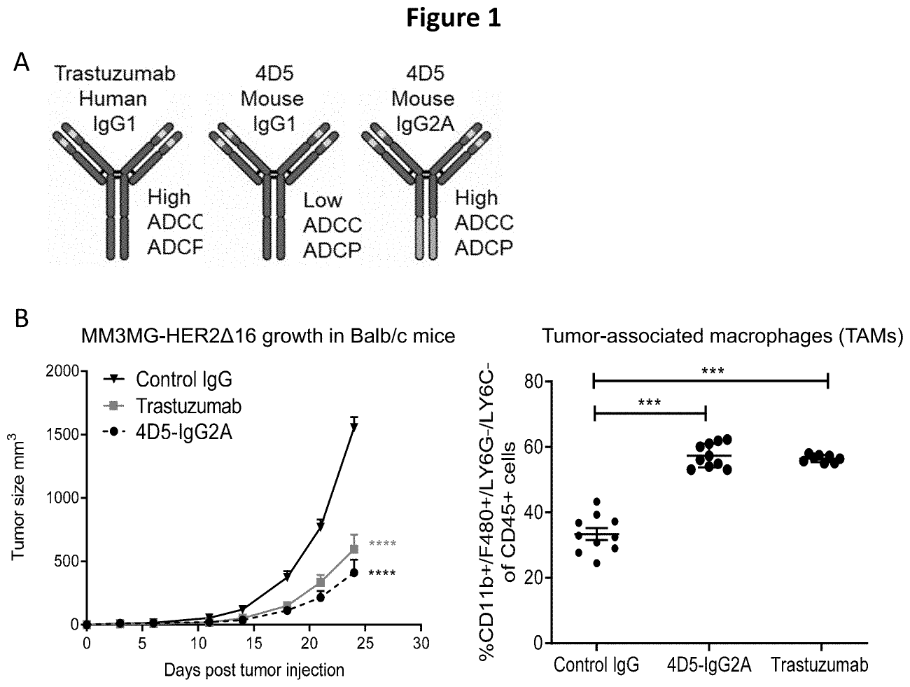

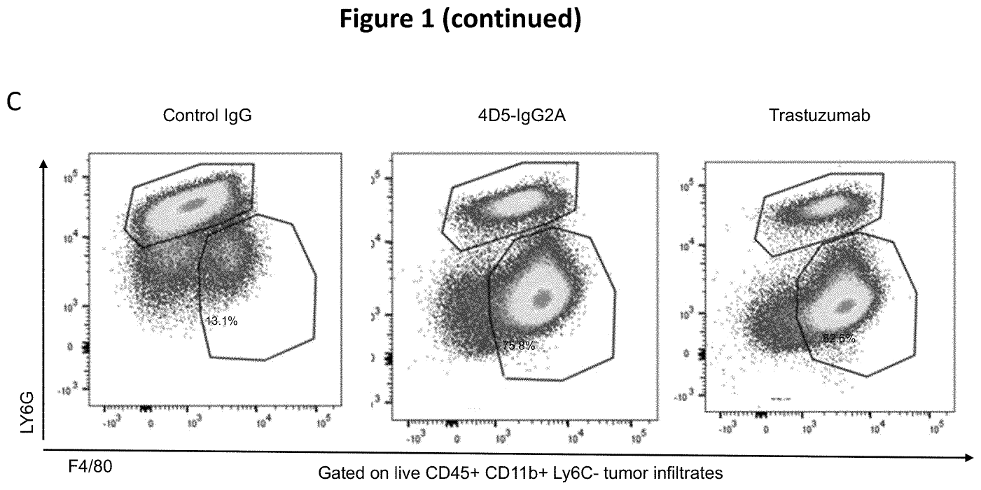

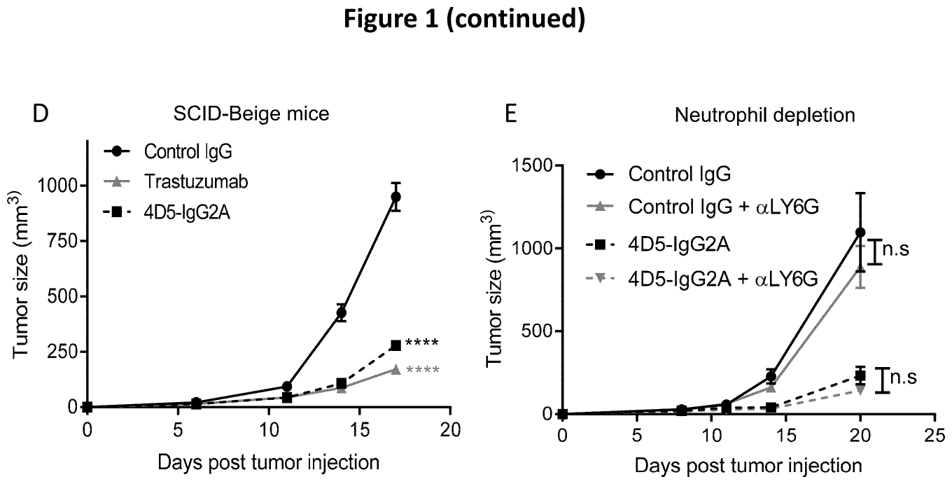

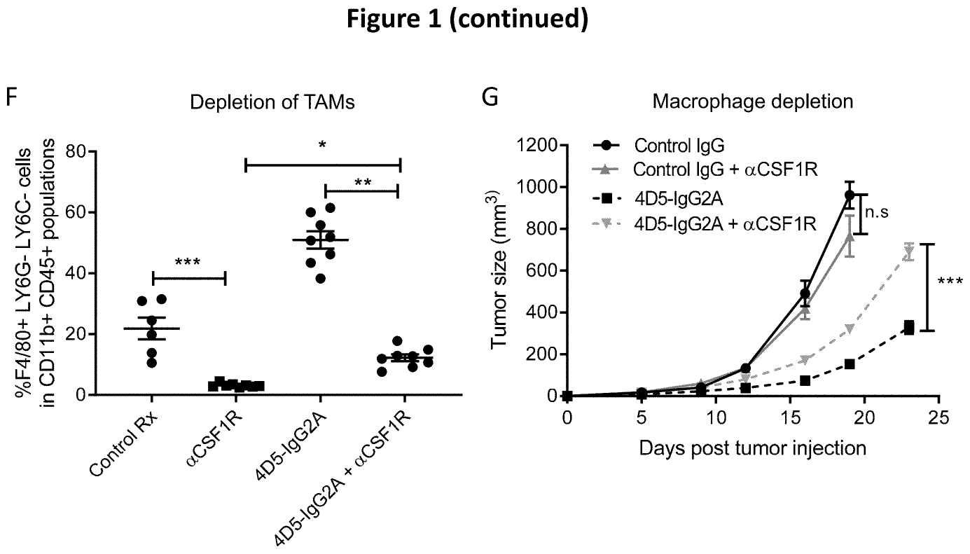

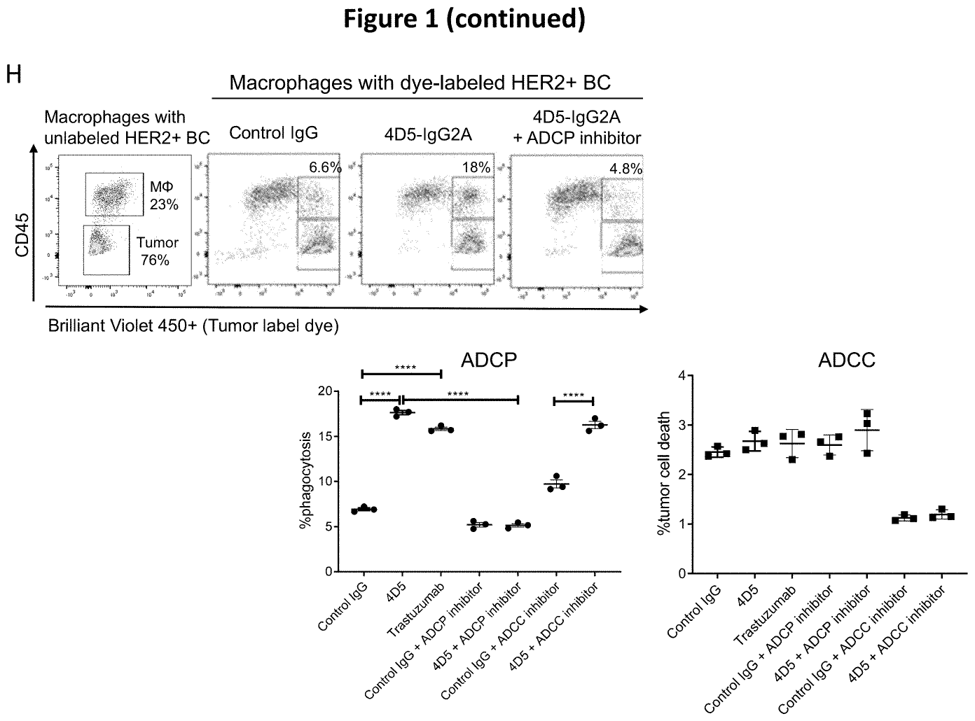

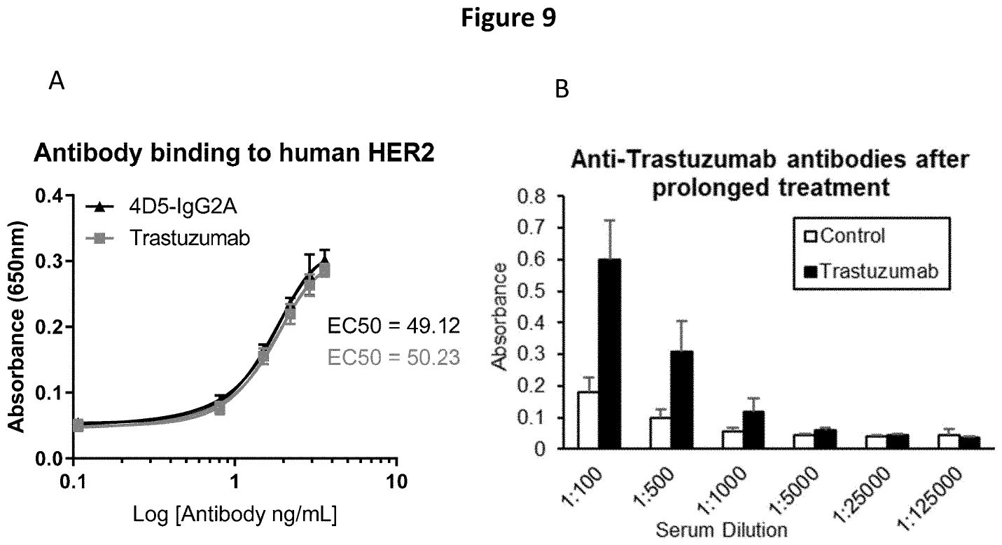

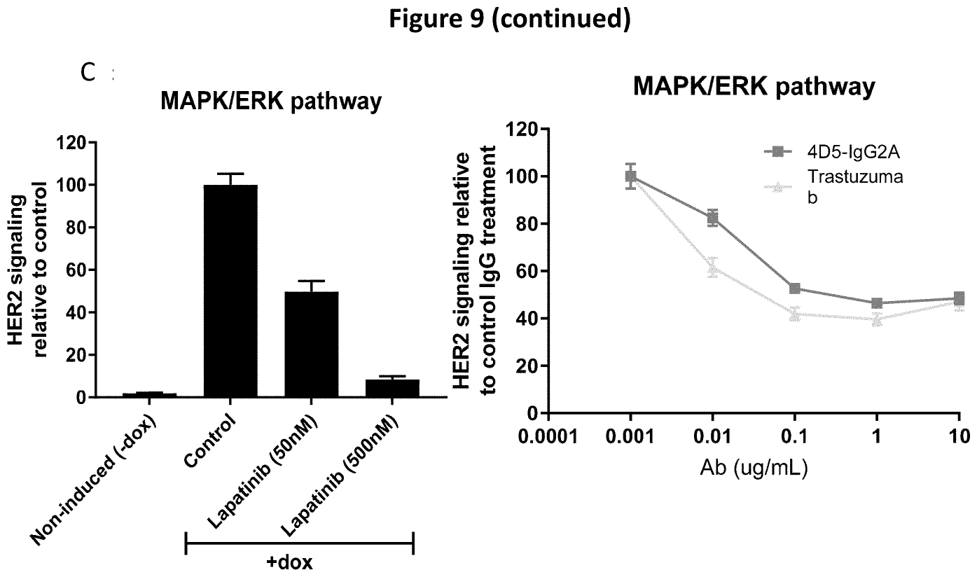

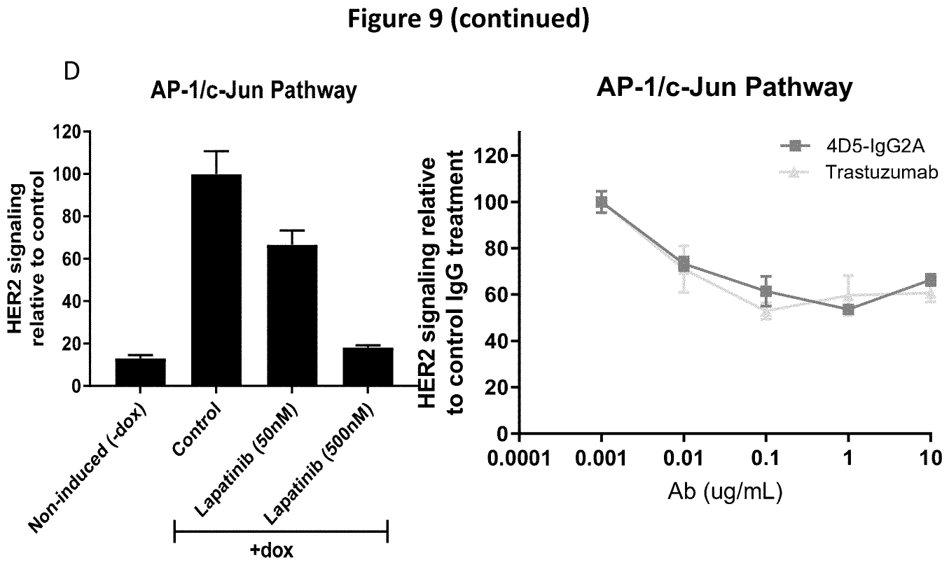

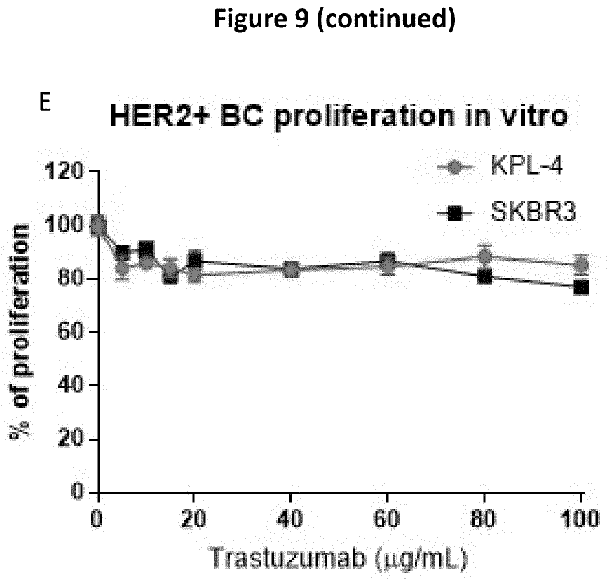

Trastuzumab was based on a HER2-specific mouse IgG1 monoclonal antibody (4D5-IgG1, low A/I ratio), which was subsequently ‘humanized’ to a human IgG1 isotype (high A/I ratio) that allows for superior activation of Fc receptors (27). Thus, to accurately study the function of Trastuzumab in an immunocompetent mouse model, we constructed a murine 4D5 monoclonal antibody, but using the IgG2A isotype (4D5-IgG2A, high A/I ratio, FIG. 1A) to better approximate an Fc-receptor activating ‘murine version’ of Trastuzumab (18, 19, 28). Unsurprisingly, we found that 4D5-IgG2A HER2 binding is equivalent to Trastuzumab (FIG. 9A). This allowed us to interrogate the importance of the HER2-antibody Fc region as well as minimize the humoral immune responses against Trastuzumab, a human antibody, when administered into a murine host (FIG. 9B).

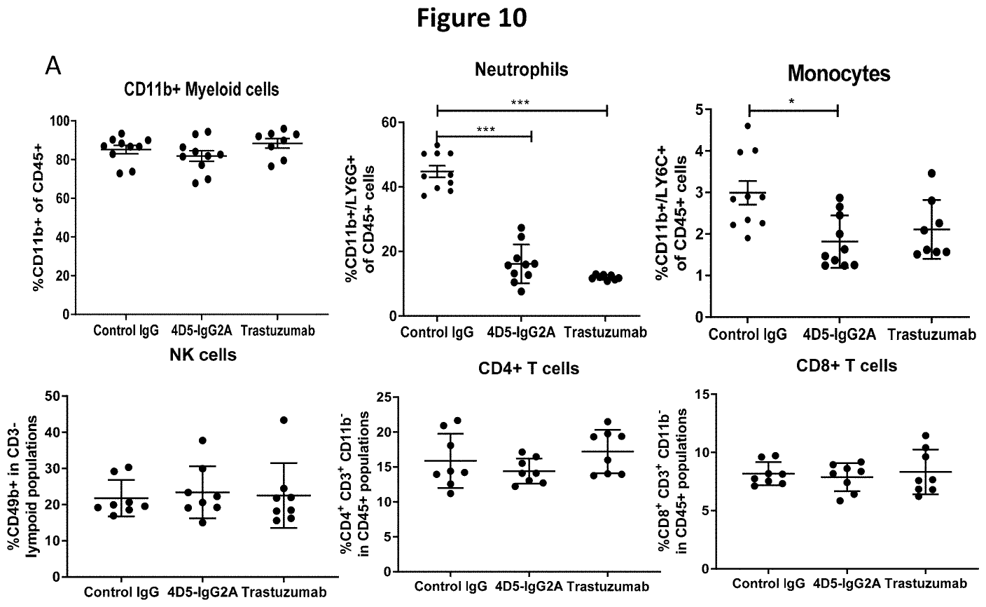

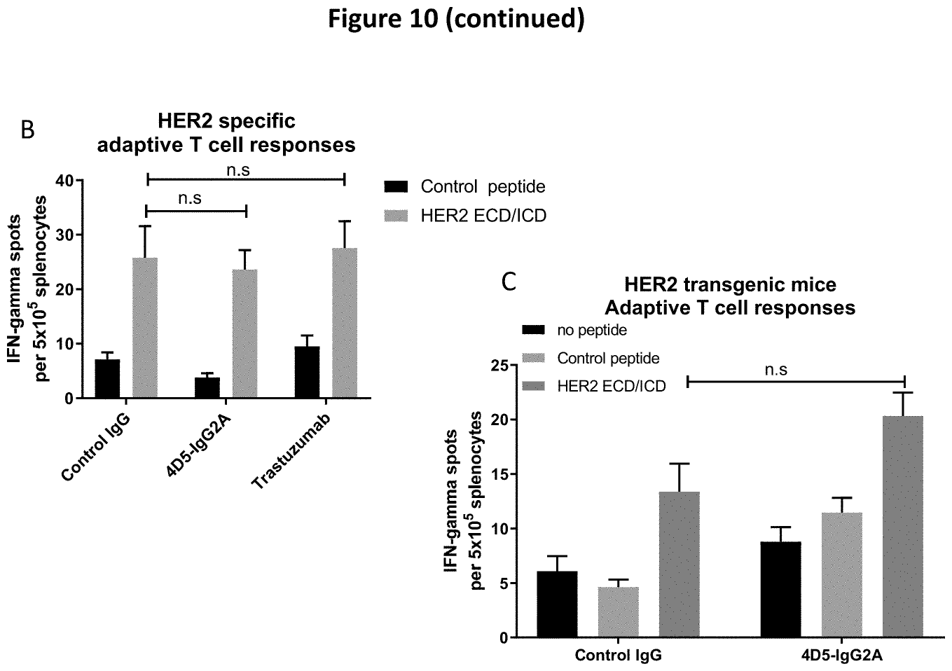

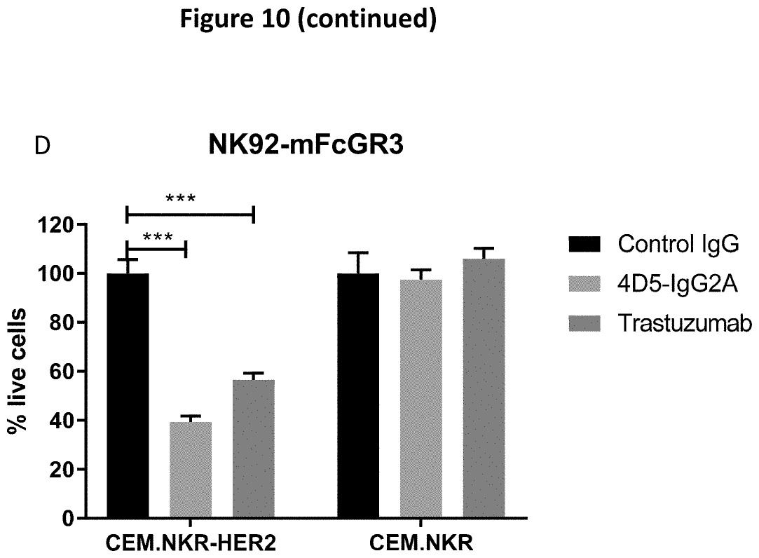

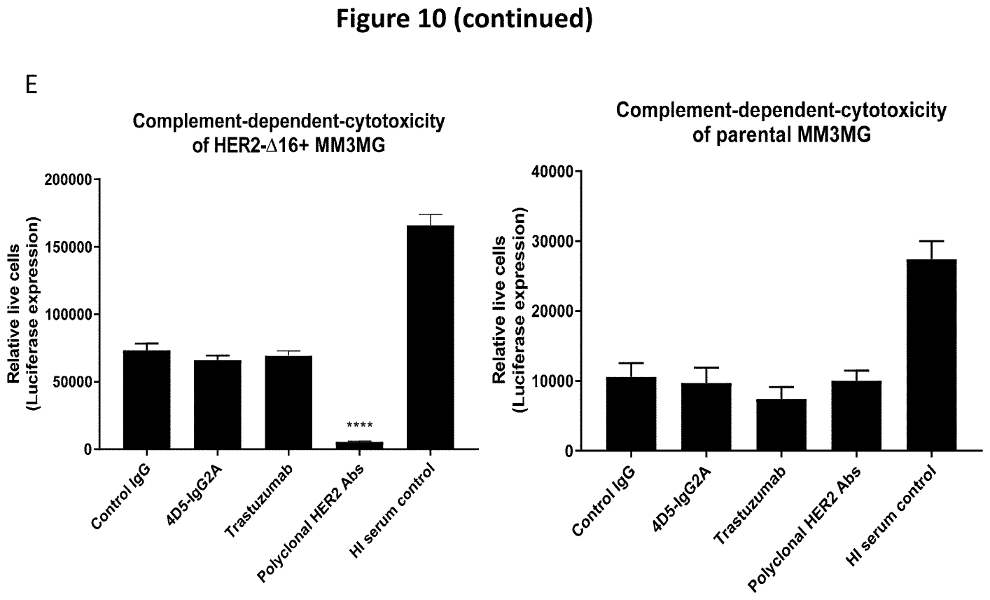

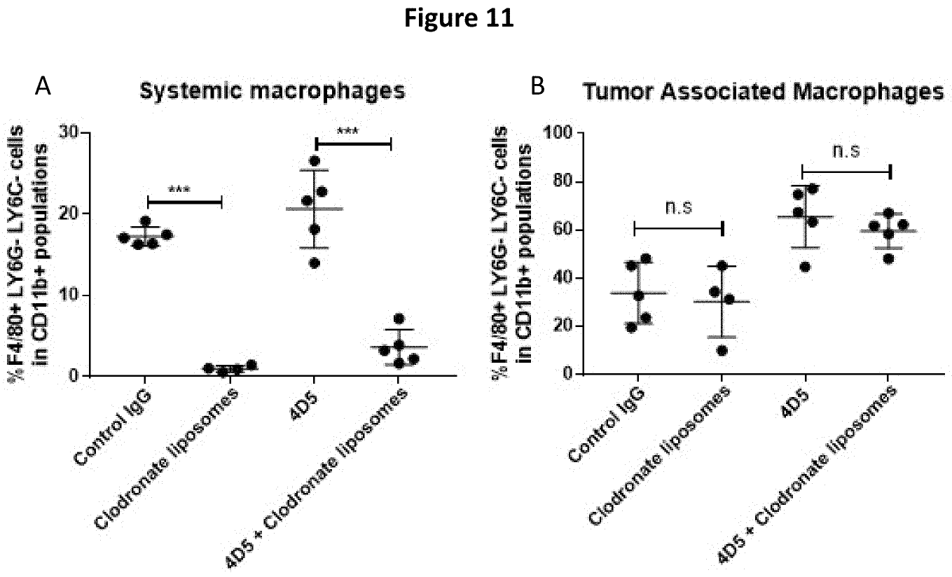

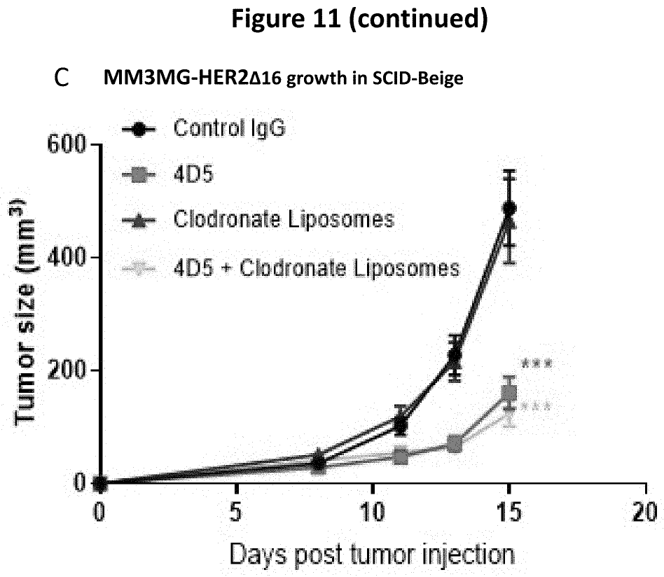

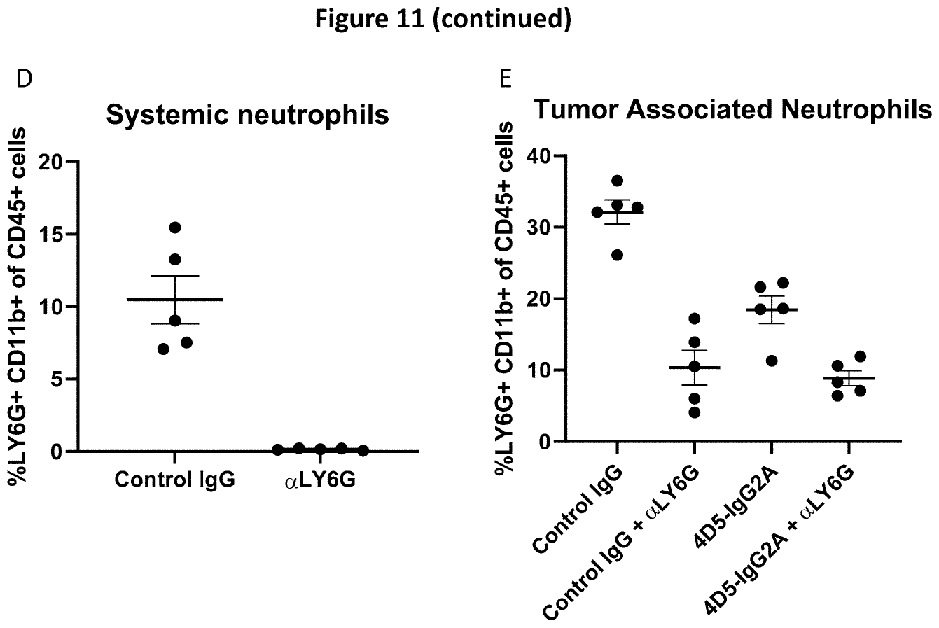

To test the antitumor efficacy of 4D5-IgG2A, we began by interrogating its impact on oncogenic HER2 signaling. As HER2 is weakly transformative in most cell lines, we employed a highly oncogenic isoform of human HER2 (HER2Δ16) that constitutively dimerizes to create a transformed BALB/c mammary cell line dependent upon HER2 signaling (21). In studies using HER2Δ16, we observed that both 4D5-IgG2A and Trastuzumab could suppress HER2 signaling (although not as potent as Lapatinib (FIG. 9C-D), but not significantly enough to prevent tumor cell growth in vitro (FIG. 9E). This is in line with several recent studies, suggesting that the impact of Trastuzumab is mediated through immune based mechanisms (29, 30). Using transformed MM3MG-HER2Δ16 as a model for HER2-driven BC growth in vivo, we next implanted these cells in the mammary fat pad of immunocompetent BALB/c mice. Tumor bearing mice were treated weekly with 4D5-IgG2A or clinical-grade Trastuzumab to determine if they could suppress tumor growth in an immunocompetent context. We found that both 4D5-IgG2A and Trastuzumab significantly suppressed HER2+ BC growth demonstrating that murine IgG2A was capable of significant antitumor activity (FIG. 1B). Notably, we observed that 4D5-IgG2A and Trastuzumab significantly increased the levels of tumor-associated macrophages (TAMs) (FIG. 1C), but did not increase other immune infiltrates such as NK cells and T cells (FIG. 10A). Furthermore, using IFN-y ELISPOT assays we found 4D5-IgG2A and Trastuzumab treatment had no effect on systemic adaptive T cells responses against human HER2 epitopes (FIG. 10B-C). In agreement with published reports (12), we observed NK cell-mediated ADCC was increased by 4D5 or Trastuzumab treatment in co-culture systems (FIG. 10D). To determine if NK cells and/or adaptive immune cells mediate antitumor immunity in vivo, we next tested HER2 mAb ability to suppress HER2+ BC growth in T cell, B cell, and NK cell deficient SCID-Beige mice. Contrary to published reports (8), we found surprisingly no change in its antitumor efficacy (FIG. 1D), suggesting the roles of adaptive immune and NK cells are minimal in Trastuzumab/4D5 action in our in vivo model system. As neutrophil levels (LY6G+ CD11b+) were suppressed (FIG. 10A) and previous studies have also implicated neutrophils in Trastuzumab-mediated immunity (14), we next depleted neutrophils using anti-LY6G in SCID-Beige studies (FIG. 11D-E), but did not observe any difference in antitumor efficacy (FIG. 1E). To investigate the possible role of complement dependent cytotoxicity (CDC), we performed CDC assays in vitro and found that neither 4D5-IgG2A nor Trastuzumab were able to induce CDC in comparison to polyclonal HER2 Abs, in line with other studies of Trastuzumab (31) (FIG. 10E).

The increase of TAMs levels after treatment suggested a functional role in Trastuzumab antitumor immunity. We therefore implemented several strategies to deplete macrophages in our SCID-beige HER2+ BC model. Using a prolonged anti-CSF1R antibody injection strategy (32), we achieved significant reduction of TAMs and which also limited TAM increase in 4D5-treated tumors (FIG. 1F). Importantly, the reduction of TAM levels resulted in a significant decrease of HER2 mAb therapeutic efficacy (FIG. 1G). We also utilized clodronate liposome injection to deplete macrophages in this model, but found we could only readily deplete macrophages in systemic circulation and not those in the tumor (FIG. 11A). Interestingly, this depletion had no effect on HER2 mAb therapy (FIG. 11B), suggesting that macrophages in the mammary tumor are the major antitumor effectors. To explore the efficacy of macrophage-mediated HER2-specific antitumor activity, we established a BMDM co-culture system to investigate the relative ADCC and ADCP activity mediated by 4D5-IgG2A and Trastuzumab (33). Using Latrunculin A, an inhibitor of actin polymerization and therefore blocking phagocytosis of immune complexes (34), we revealed the dominant antitumor activity of HER2 mAbs mediated by macrophages is through ADCP (FIG. 1H). Concanamycin A, an V-ATPase inhibitor reported to also inhibit perforin and cytotoxicity (35), had no effect on HER2 mAb activities. Collectively, these results suggested that Trastuzumab therapy modifies the tumor microenvironment by promoting TAM expansion, and that the dominant mechanism of action by Trastuzumab is mediated by ADCP of HER2+ tumor cells by macrophages.

### The ADCP Activity of 4D5 Requires the Engagement with Fcγ-Receptors and is Isotype Dependent

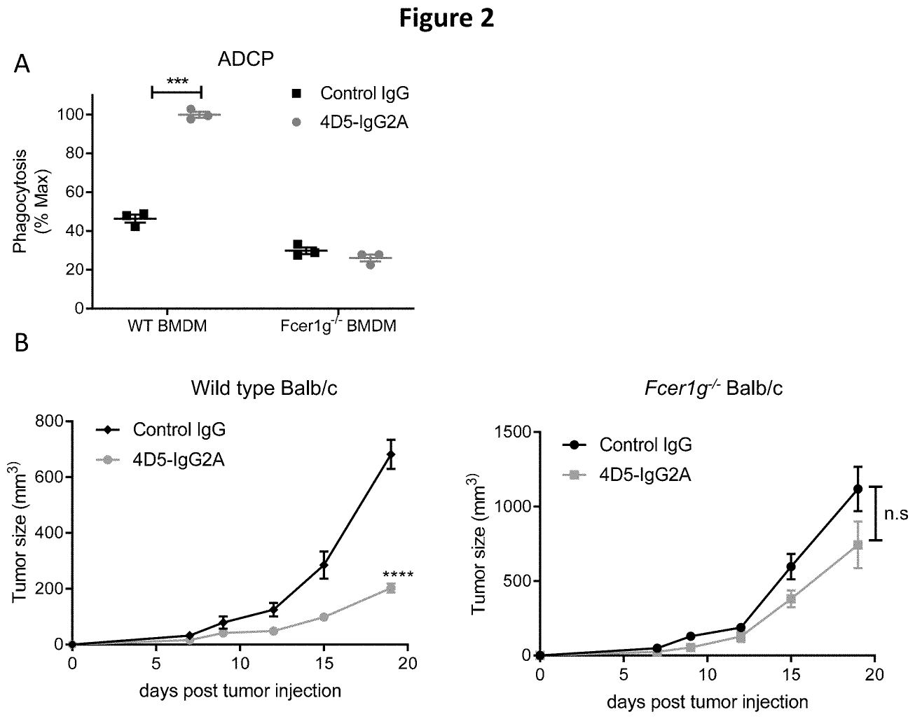

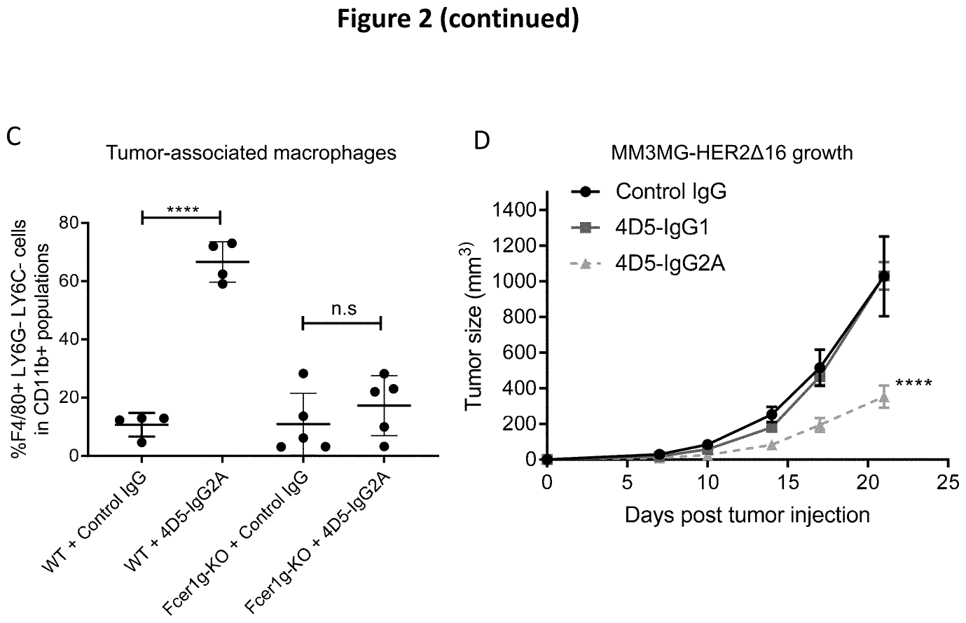

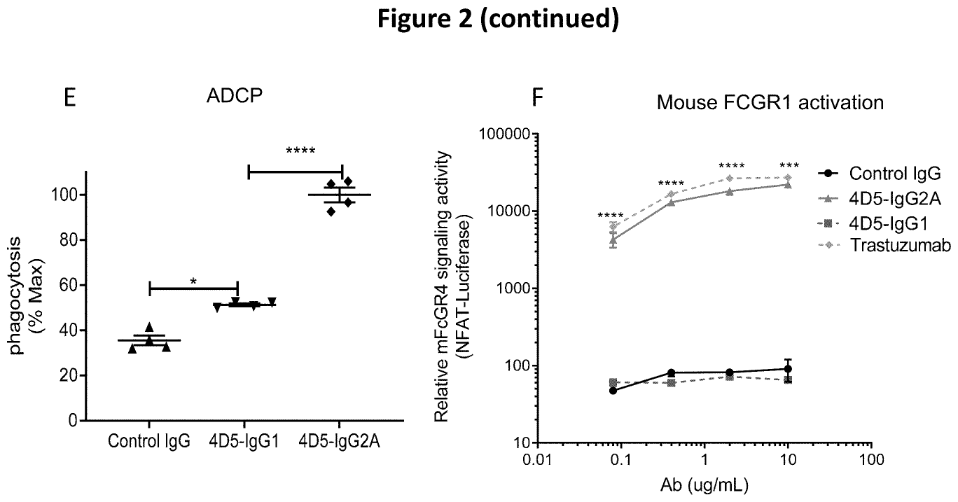

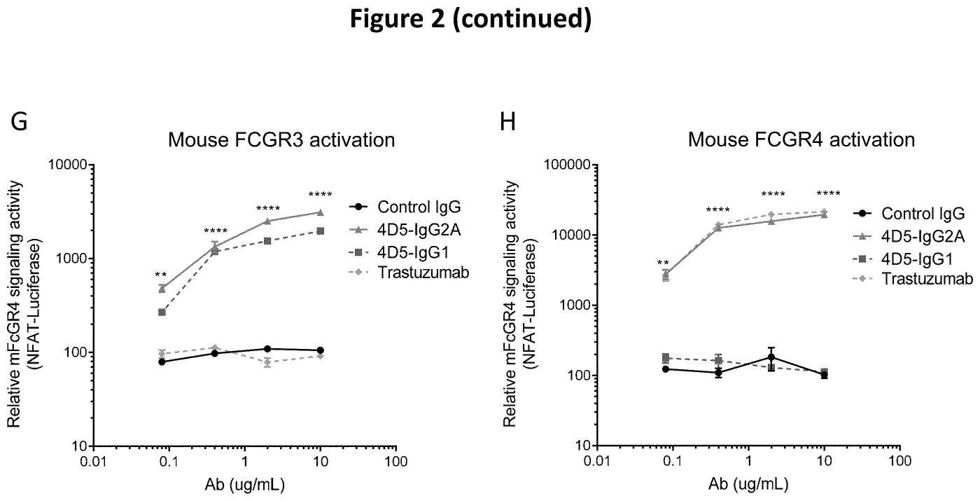

To further validate the mechanism of ADCP by 4D5-IgG2A treatment, we utilized Fcer1g^{-/-} animals to test the requirement for Fcγ-receptor (FCGR) engagement on phagocytic immune cells. Using macrophages cultured from Fcer1g^{-/-} and control mice, in vitro ADCP assays revealed that FCGRs on macrophages are critical for 4D5-induced ADCP of HER2+ BC (FIG. 2A). Accordingly, we found the in vivo antitumor efficacy of 4D5-IgG2A therapy are mostly ablated in Fcer1g^{-/-} mice (FIG. 2B). Importantly, FCGR expression was also required for macrophage expansion by 4D5-IgG2A in the tumor microenvironment (FIG. 2C).

These data demonstrated that HER2 mAb engagement with macrophage FCGRs is required for ADCP activity. Among the four mouse FCGRs, FCGR4 is the predominant FCGR mediating macrophage ADCP, plays a central role for mouse IgG2A activity, and has also been shown to exhibit the strongest binding affinity for Trastuzumab (16, 36-38). To determine the impact of HER2-mAb isotype on FCGR4 engagement and antitumor efficacy, we compared the efficacy of 4D5-IgG1 (low A/I ratio) and compared its antitumor efficacy with 4D5-IgG2A (FIG. 1A). We found that unlike 4D5-IgG2A which elicited significant antitumor effects in vivo and ADCP in vitro, 4D5-IgG1 has no effect against HER2+ BC in vivo (FIG. 2D) and was inferior in promoting tumor ADCP by BMDM

(FIG. 2E). To determine their impact on FCGR4 and other activating FCGRs directly, we developed a mouse FCGR activation and signaling to NFAT-luciferase reporter system based on published methods (39). In agreement with established literatures on mouse IgG subclasses and FCGR biology (18, 19, 40), we found that 4D5-IgG2A engages with all three activating FCGRs, whereas 4D5-IgG1 only weakly activates FCGR3 (FIG. 2F-2H). Additionally, mouse FCGR1 and FCGR4 have strong human-murine cross-reactivity with clinical grade human Trastuzumab (human IgG1 isotype) as reported before (40), thus potentially explaining its in vivo efficacy in mice. Collectively, these results illustrate that HER2 mAb's antitumor activity requires the successful engagement and activation of Fcγ-receptors on macrophages to induce ADCP.

### CD47 Blockade Increased Therapeutic Efficacy of 4D5 and Augments Tumor-Associated Macrophage Expansion and Phagocytosis

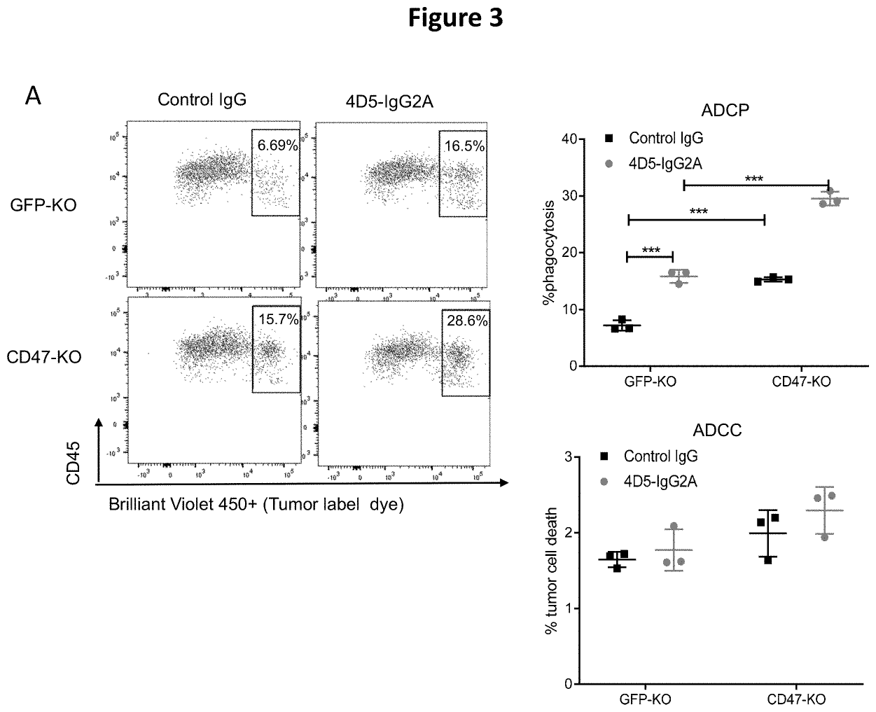

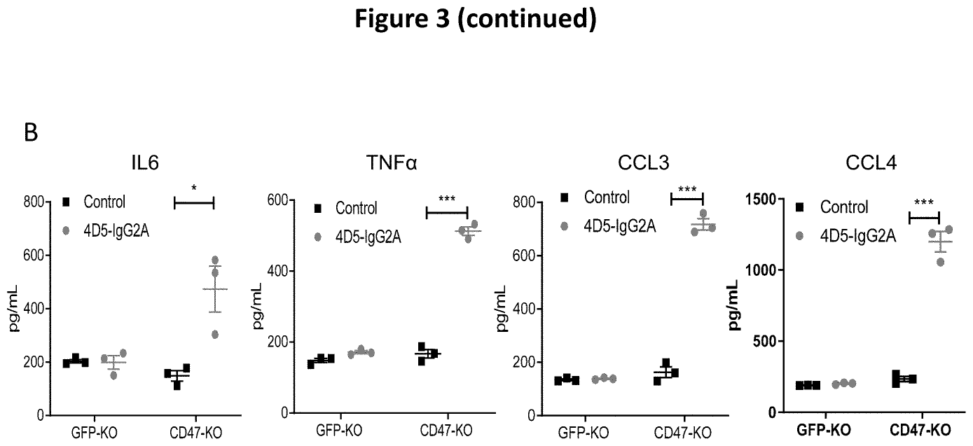

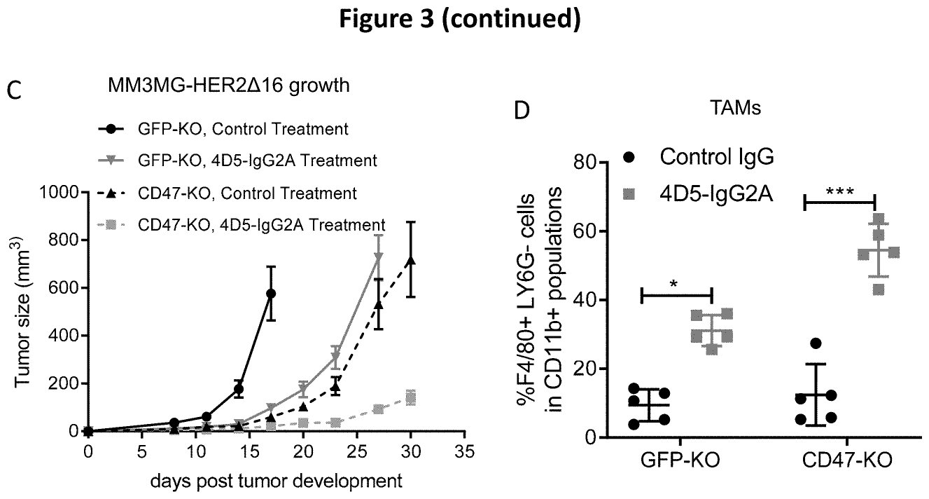

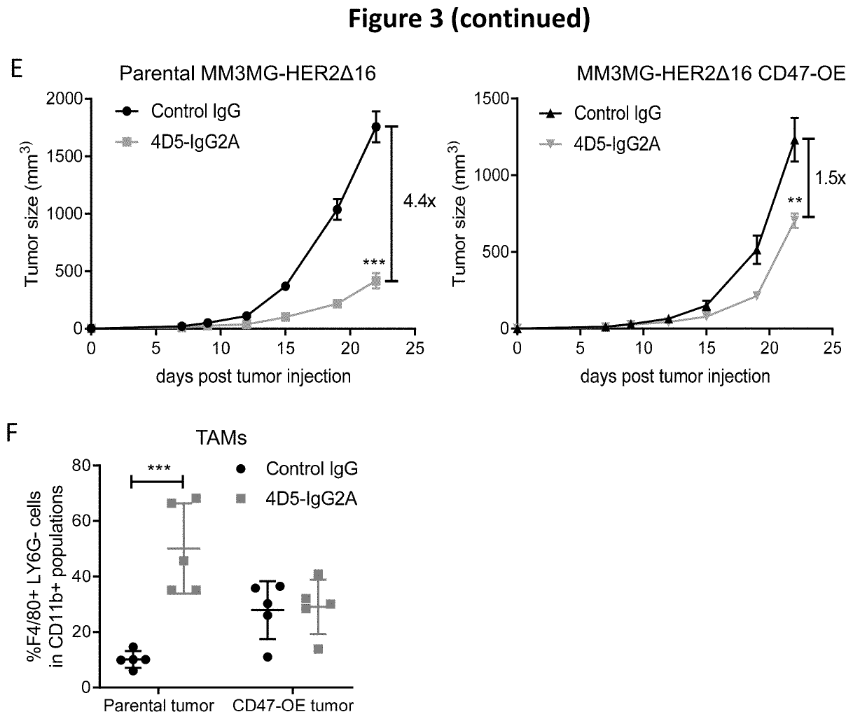

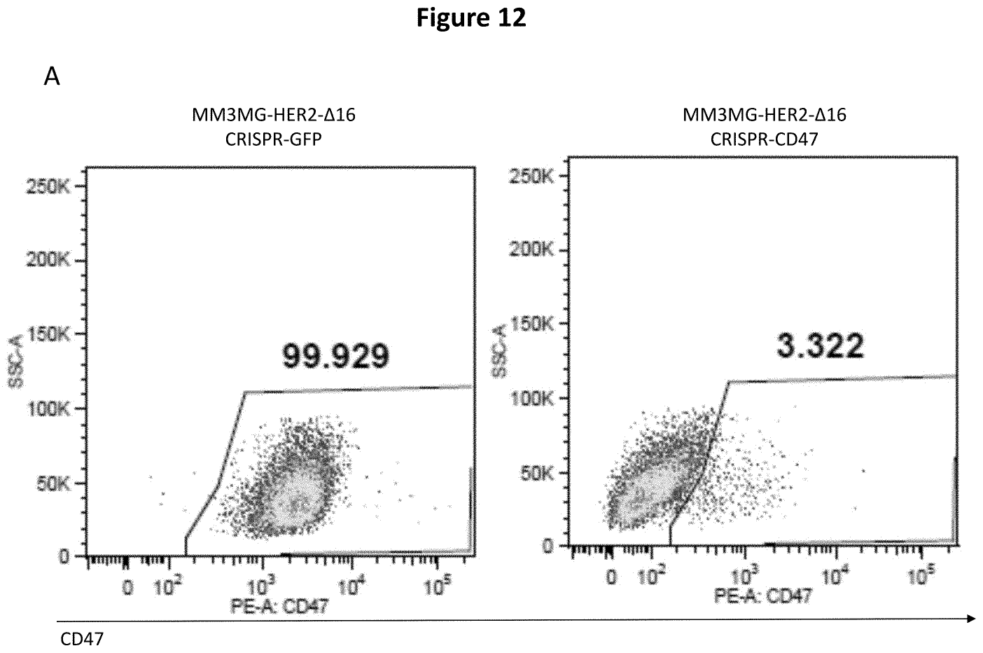

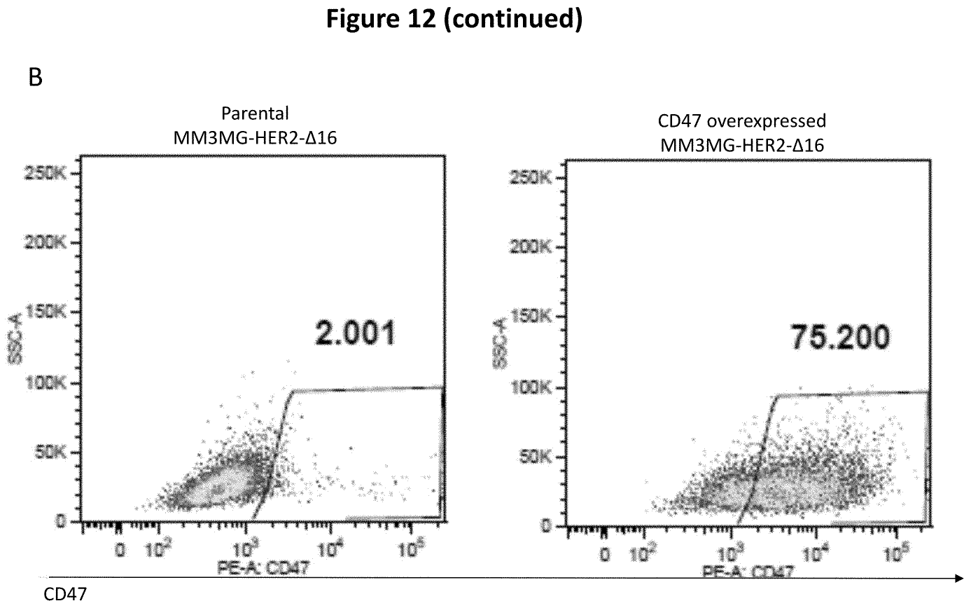

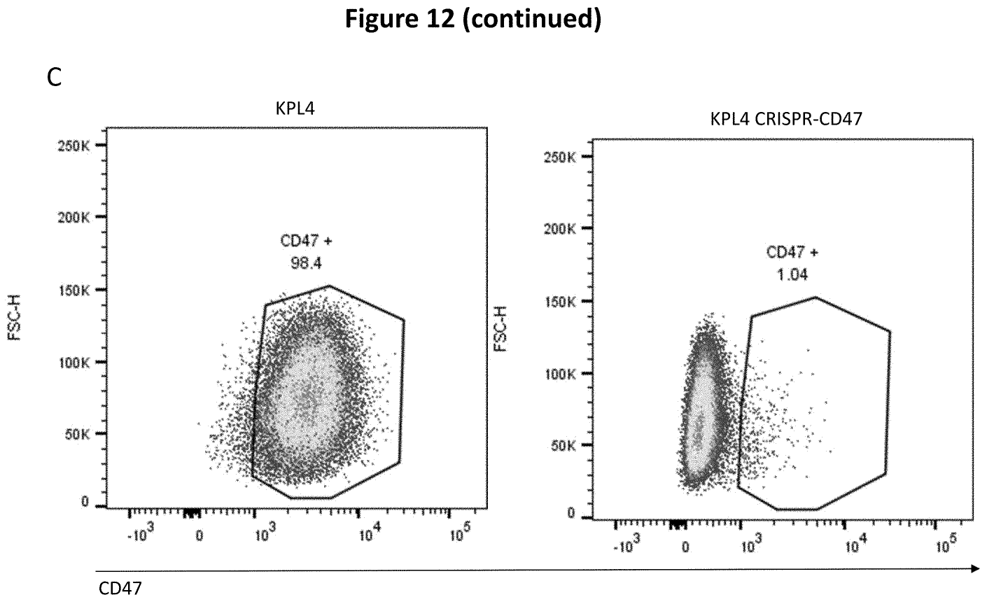

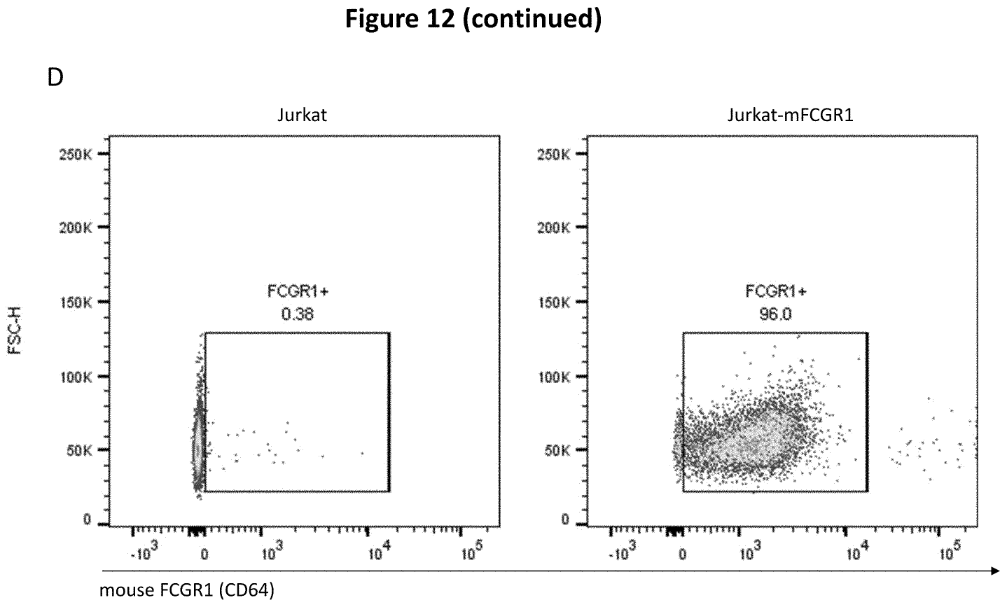

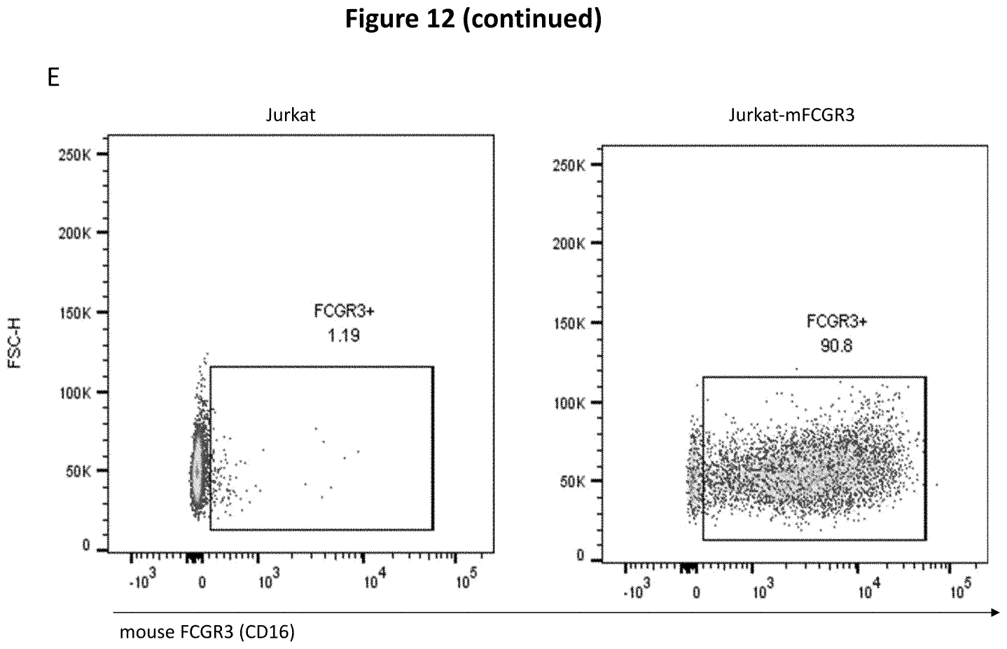

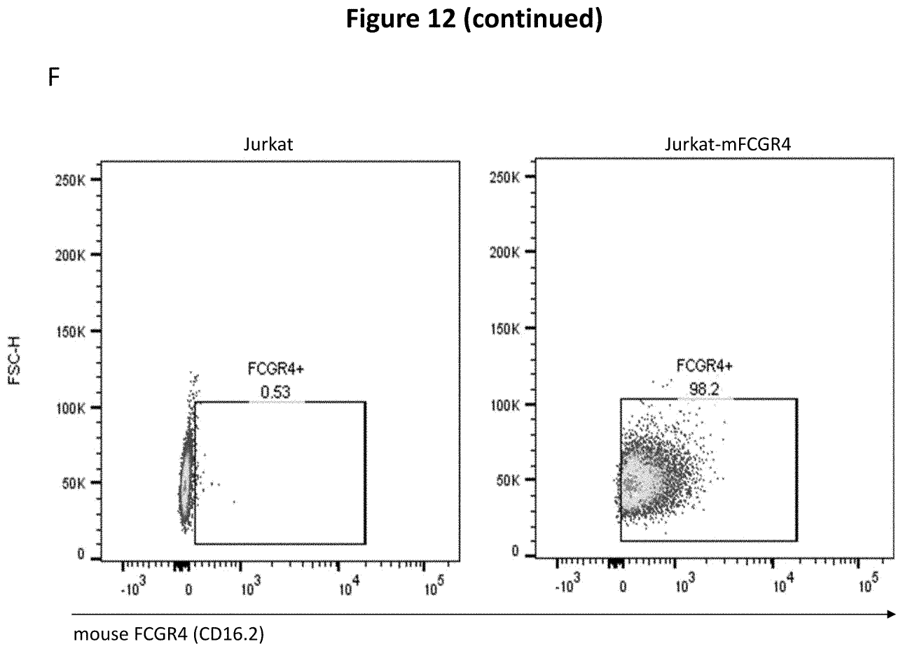

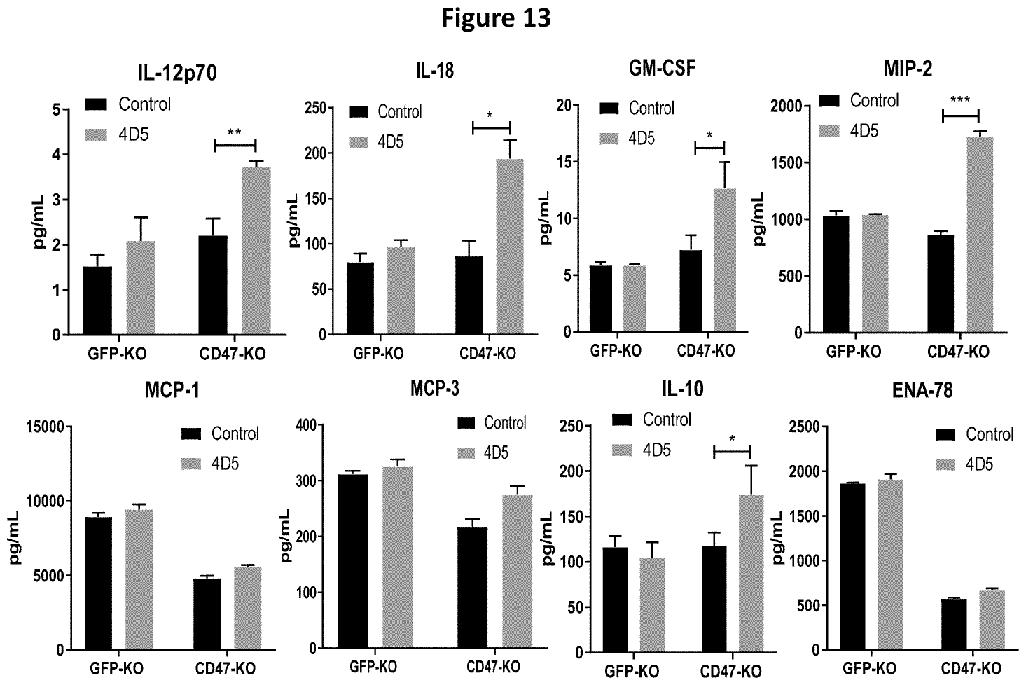

Our findings strongly supported an ADCP MOA for Trastuzumab antitumor efficacy, which suggests strategies to enhance ADCP may be synergistic with Trastuzumab therapies. As previous studies have demonstrated that blockade of CD47-SIRPα can enhance mAb therapeutic efficacy, we investigated if targeting this ADCP-specific axis would enhance HER2 mAb ADCP without affecting ADCC activity. To begin our investigation, we documented the elevated expression of Cd47 in our MM3MG-HER2Δ16 tumors and generated CD47-KO cells (FIG. 12A) to determine the contribution of this axis to ADCP and ADCC in vitro. We observed that CD47-KO tumor cells exhibited generally enhanced ADCP that was significantly enhanced by HER2 mAbs, but had no effect on ADCC (FIG. 3A). Additionally, we found that 4D5-mediated ADCP of CD47-KO tumors elicited the expression of pro-inflammatory cytokines and chemokines by macrophages (e.g. IL6, TNFα, CCL3, CCL4 etc.), presumably due to enhanced ADCP activity (FIG. 3B and FIG. 13). This demonstrates that 4D5-IgG2A alone triggers ADCP but was insufficient to stimulate significant pro-inflammatory activation within macrophages. However, upon blockade of the CD47 negative regulatory axis, ADCP and an associated pro-inflammatory phenotype was significantly enhanced in macrophages.

As CD47 directly altered ADCP and macrophage activation in vitro, we next evaluated the impact of CD47-KO expression on tumor growth and HER2 mAb therapy in vivo. We found that CD47-KO HER2+BC cells showed a delayed growth when implanted into mice, and were significantly more susceptible to 4D5-IgG2A inhibition (FIG. 3C). Furthermore, we found significantly elevated TAM levels in CD47-KO tumors compared to the control tumors after 4D5-IgG2A treatment (FIG. 3D). In a reciprocal approach, we overexpressed Cd47 in the tumor cells (FIG. 12B) and found this increased tumor resistance to 4D5-IgG2A therapy (FIG. 3E) and prevented TAMs increase (FIG. 3F). These two genetic approaches validated the role of CD47 in suppressing Trastuzumab ADCP-mediated antitumor activity, and suggest blockade of CD47 could unleash the full potential of Trastuzumab therapeutic efficacy by altering macrophage activation and expansion.

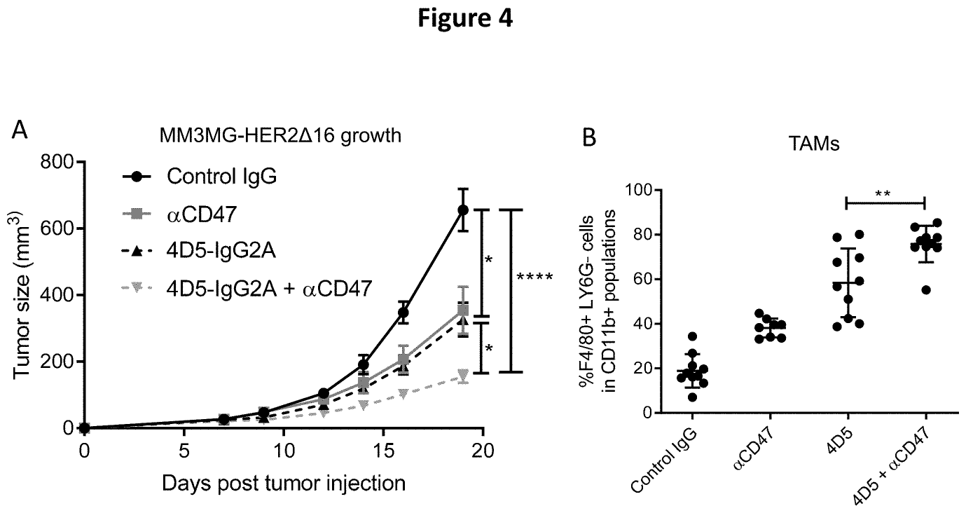

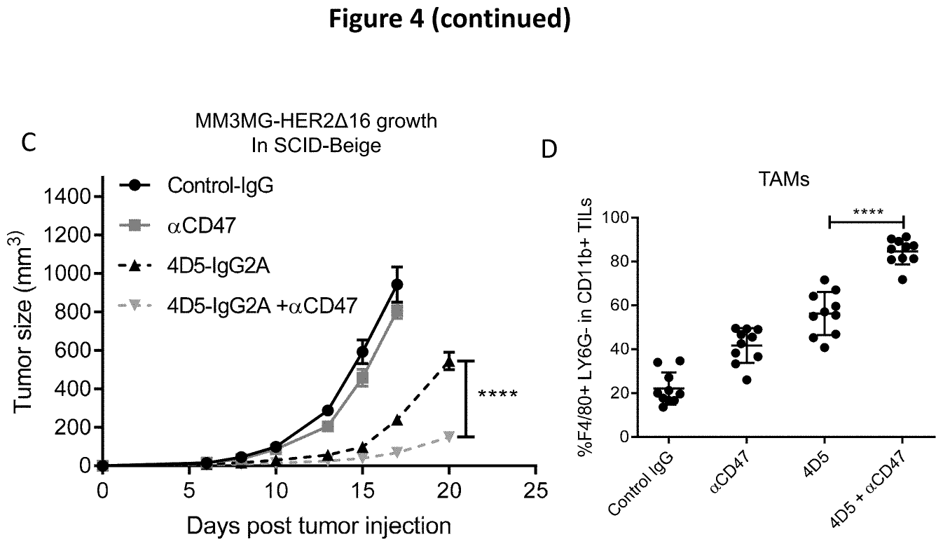

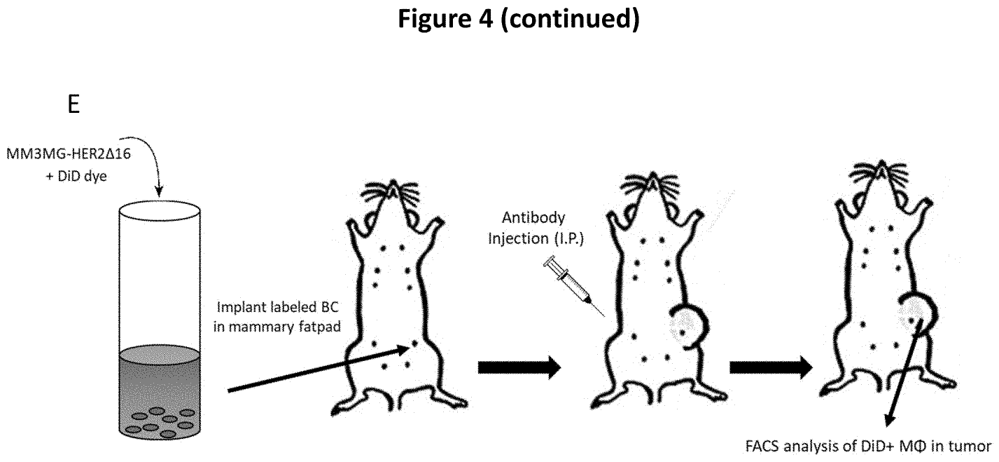

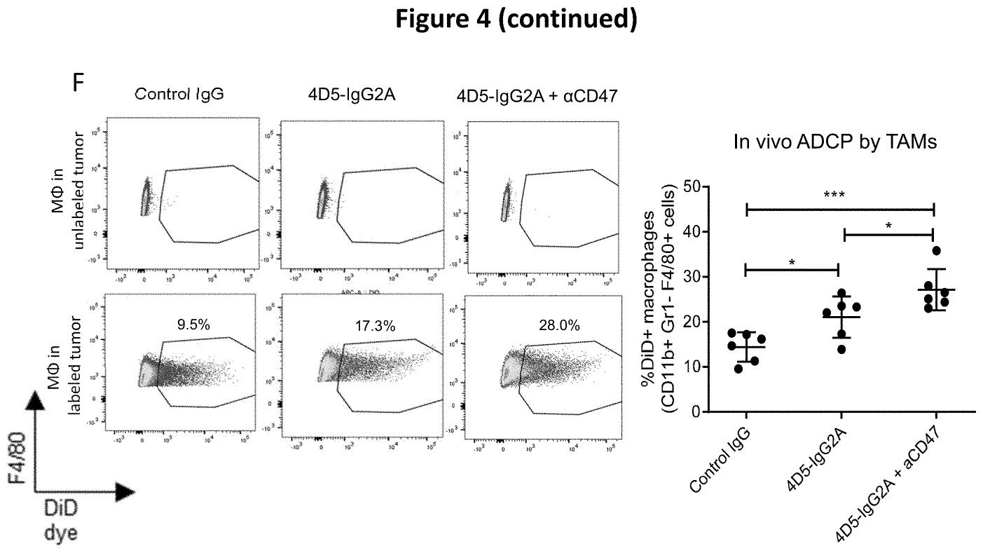

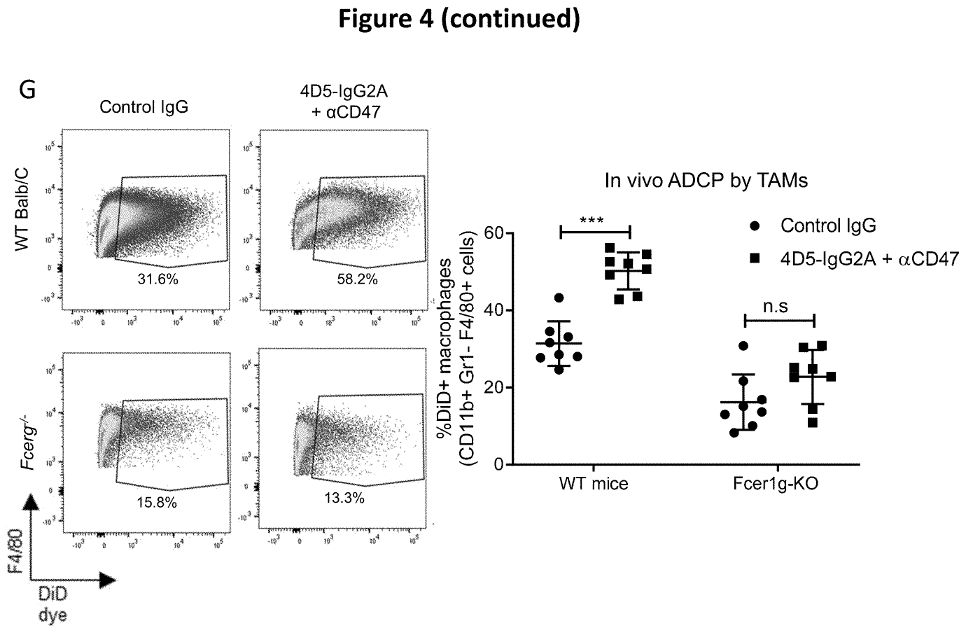

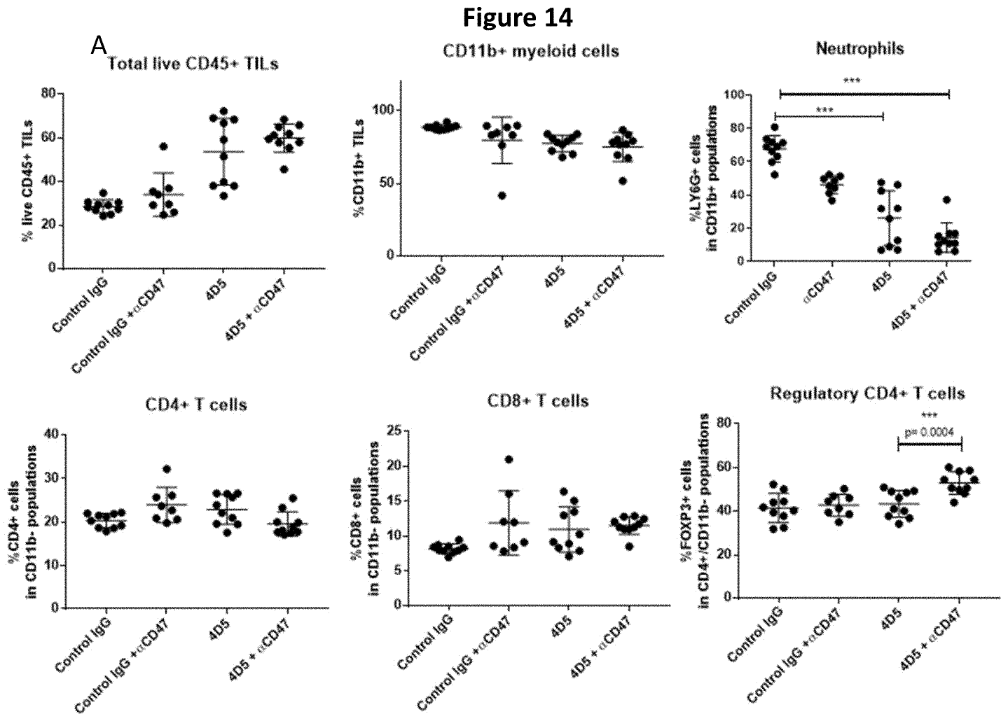

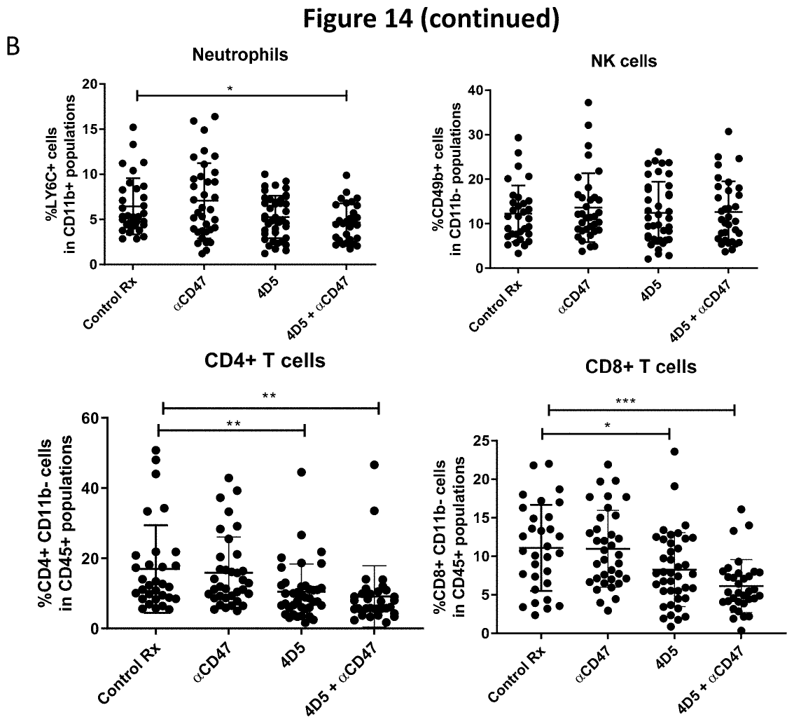

As recent studies have suggested CD47 blockade antibodies can elicit clinical responses (41), we next wanted to determine if CD47 blockade may enhance Trastuzumab efficacy. Thus, we combined 4D5-IgG2A mAb with CD47 blockade antibody MIAP410 in immunocompetent mice bearing the MM3MG-HER2416 tumors. While 4D5-IgG2A and CD47 blockade monotherapies both showed therapeutic efficacy, their combination significantly suppressed tumor growth more effectively than either 4D5-IgG2A or CD47 alone and also further increased TAM levels (FIG. 4A and 4B and S7). In contrast, we observed that levels of other infiltrating immune cell types, except for regulatory T cells, were not significantly increased by weekly treatment of 4D5-IgG2A with CD47 blockade (FIG. 14A). As regulatory T cells were altered, we speculated that adaptive immune responses could also play a role in these enhanced responses. To explore the impact of adaptive immunity in the context of CD47 blockade, we repeated our in vivo experiments in adaptive immune-deficient SCID-Beige mice (FIG. 4C). As before, we observed a strong combinatorial effect between HER2 mAb and CD47 blockade, suggesting adaptive immunity and NK cells were not essential to the enhanced response with this combination therapy. Also as before, we found that CD47 blockade with 4D5-IgG2A further increased TAMs levels (FIG. 4D), suggesting that relieving the CD47 checkpoint specifically promotes macrophage expansion and phagocytosis in tumors.

In order to directly demonstrate tumor ADCP by endogenous macrophages in the tumor microenvironment, we labeled MM3MG-HER2Δ16 tumor cells with DiD dye (a carbocyanine mernbrane-binding probe) prior to implantation, a strategy to detect phagocytosis of labeled target cells in vivo (42). When the tumors reached a volume of ˜1000 mm³, we treated the animals with 4D5-IgG2A antibody or in combination with CD47 blockade (FIG. 4E). FACS analysis showed increased phagocytosis of labeled tumor cells by TAMs in 4D5-IgG2A treated animals (FIG. 4F), directly demonstrating 4D5-IgG2A treatment promotes ADCP of HER2+ tumor cells in vivo. Furthermore, we found the addition of CD47 blockade further increased ADCP of labeled tumor cells by TAMs (FIG. 4F). As expected, this therapeutic mechanism requires the engagement with FCGRs on macrophages, since 4D5+αCD47 induced ADCP of tumor cells in vivo was completely abolished in Fcer1g-KO mice (FIG. 4G). In sum, these studies demonstrate that HER2 mAb stimulates ADCP from endogenous TAMs against HER2+ BC, which can be boosted via combination with CD47 blockade therapy.

### CD47 Blockade Synergizes 4D5 Therapeutic Activity in a Transgenic HER2+Breast Cancer Mouse Model

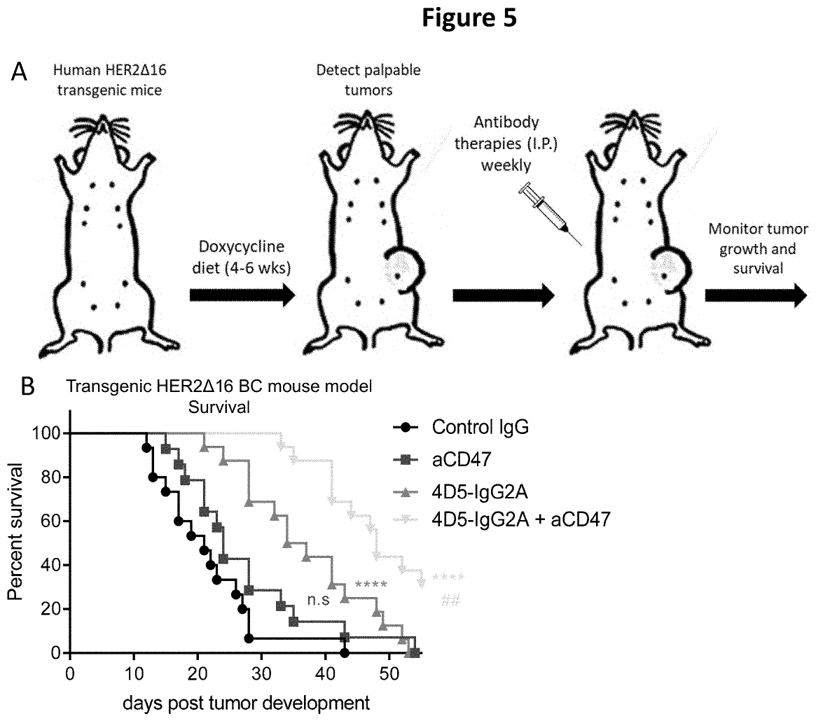

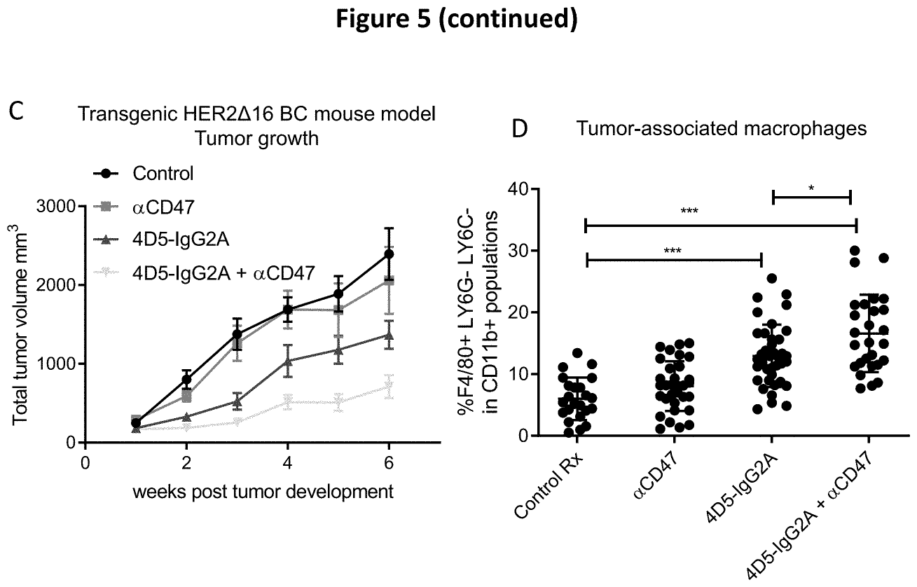

Having demonstrated efficacy in an orthotopic model of HER2+ BC, we wanted to extend our study using a spontaneous model of HER2+ BC that approximates a late stage HER2+ BC (where HER2 mAbs are not highly effective) (43). Analogous to a clinical trial (FIG. 5A), the individual animals with palpable breast tumors (˜200 mm³) were enrolled in a specific treatment group. We found that mice in the 4D5-IgG2A monotherapy treatment group had a significant increase in survival time and delayed tumor growth, whereas CD47 blockade monotherapy had no significant effect compared to the control group (FIGS. 5B and 5C). Strikingly, combination therapy of 4D5-IgG2A with CD47 blockade resulted in a further prolonged survival rate and delayed tumor growth compared to 4D5 monotherapy, suggesting that this combination may be efficacious in advanced HER2+ BCs. To determine if these therapies again alter the immune infiltrates, we analyzed the composition of the tumor microenvironment by flow cytometry. As before, we found an increase in TAMs within the 4D5-IgG2A monotherapy group, whereas the combination therapy group showed an even higher increase (FIG. 5D). Additionally, we also observed a slight reduction of T cell infiltration and neutrophil levels (FIG. 14B).

### Single-Cell Transcriptome Analysis of TAMs in HER2+ BC After 4D5 with CD47 Blockade Combination Therapy

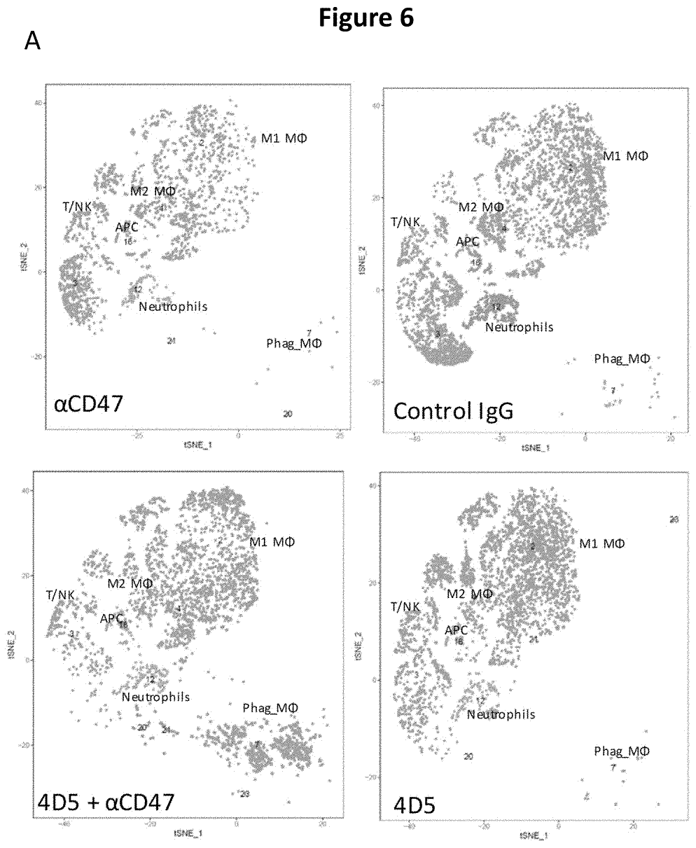

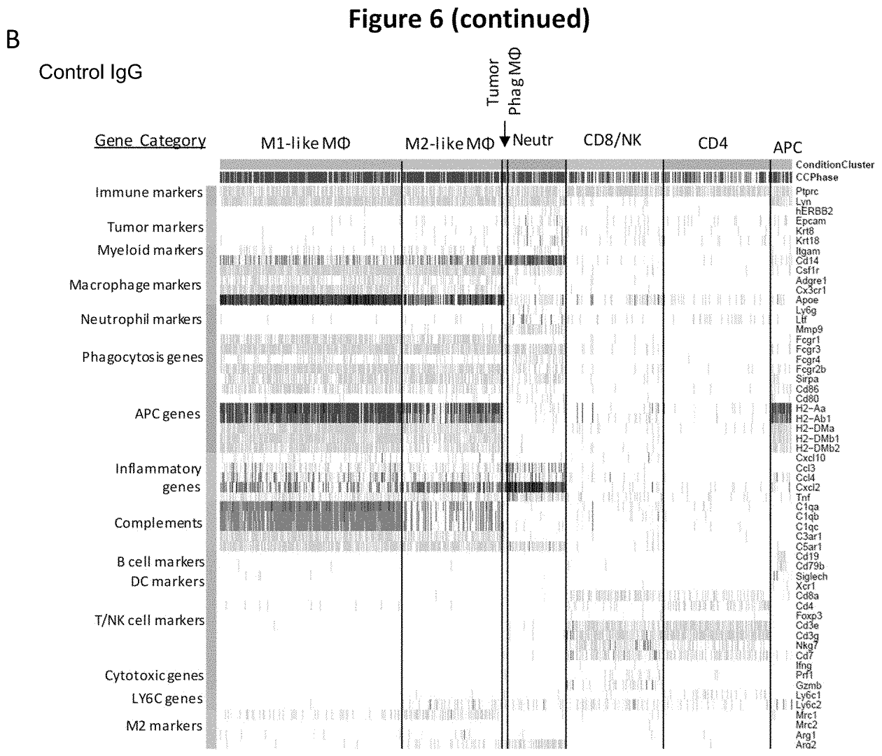

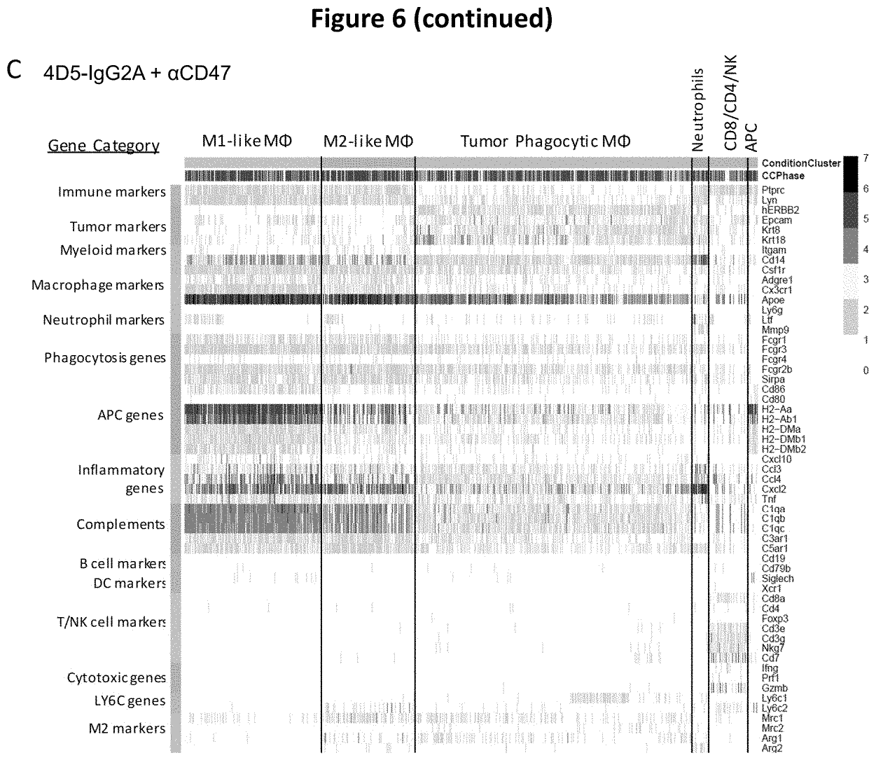

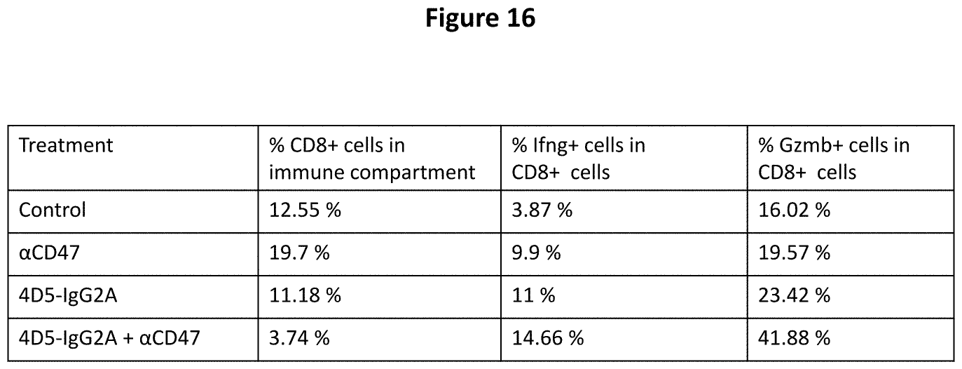

To further determine if macrophages were differentially activated, we performed single-cell RNA sequencing on dissociated tumors from the HER2 transgenic mice. These studies confirmed the increase of macrophages upon 4D5-IgG2A plus αCD47 treatment and also revealed the emergence of a distinct group of macrophages (that we termed “Phag MΦ” cluster) that are phenotypically distinct from the resident macrophage clusters (i.e. “M1 MΦ” and “M2 MΦ” clusters) (FIGS. 6A and 6B). Notably, we found that this Phag MΦ cluster contained large quantities of human HER2 RNA and other tumor specific transcripts (such as Epcam and Cyto-keratins), indicating that they have actively phagocytosed tumor cells. This cluster was expanded by 4D5-IgG2A treatment and increased further by combination 4D5+CD47 mAbs treatment (FIG. 6B and Table 1). In agreement with our FACS analysis, the level of total macrophages were significantly increased while T cell and neutrophil levels were reduced after 4D5 or combination therapy (FIG. 6B and Table 1). Interestingly, the frequency of cytotoxic gene expression (Ifng and Gzmb) among CD8+ T cells were increased following treatments (FIG. 6 and Table S1 (FIG. 16)).

TABLE 1

| | | | | Tumor Phagocytic |
|---|---|---|---|---|
| | | Average | | MΦ |
| | # of | length on | | (% MΦ containing |
| | tumors | treatment | Total MΦ | hERBB2 |
| Treatment | analyzed | regimen | cluster size | transcripts) |
| Control | 3 | 28 days | 2354/4527 | 4.22% |
| | | | (52%) | |
| αCD47 | 2 | 36 days | 1228/2154 | 3.95% |
| | | | (57%) | |
| 4D5-IgG2A | 4 | 45 days | 2938/3815 | 9.07% |
| | | | (77%) | |
| 4D5-IgG2A + | 4 | 56 days | 4673/5079 | 48.44% |
| αCD47 | | | (92%) | |

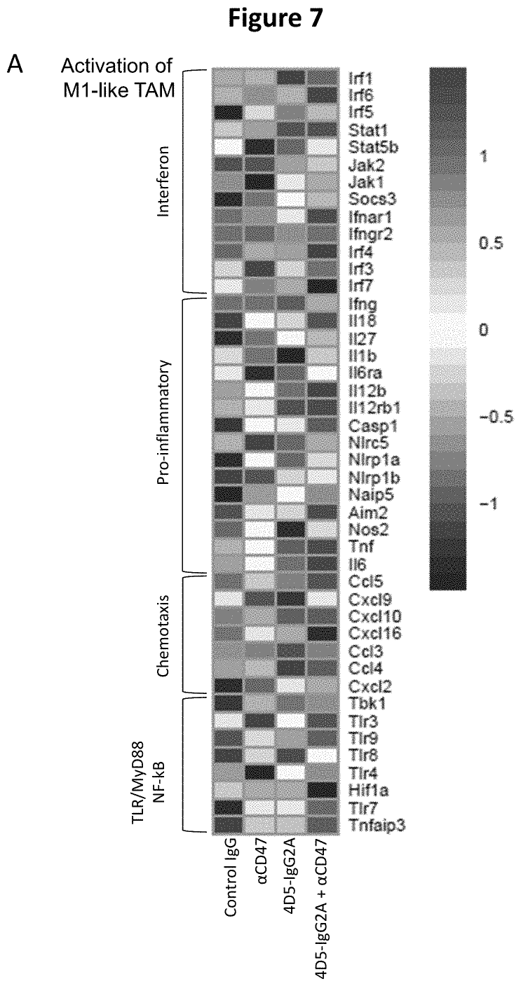

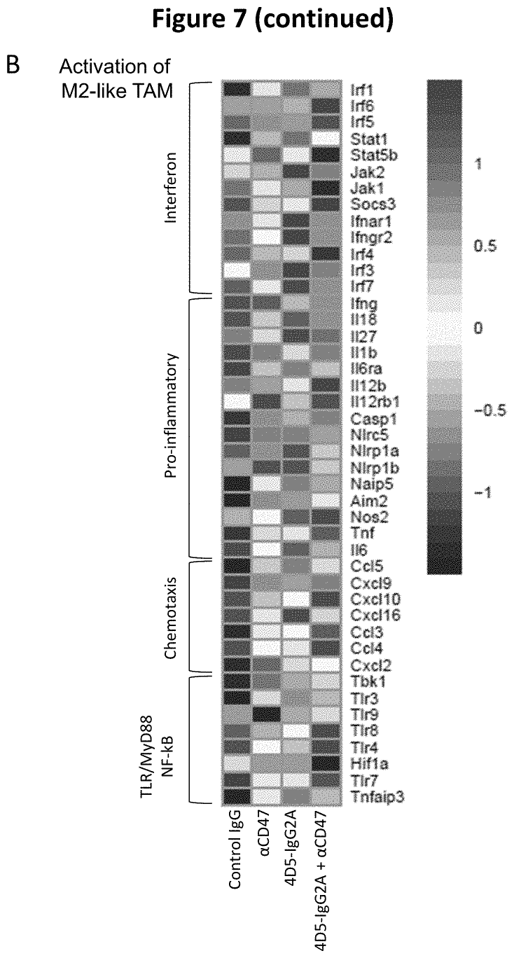

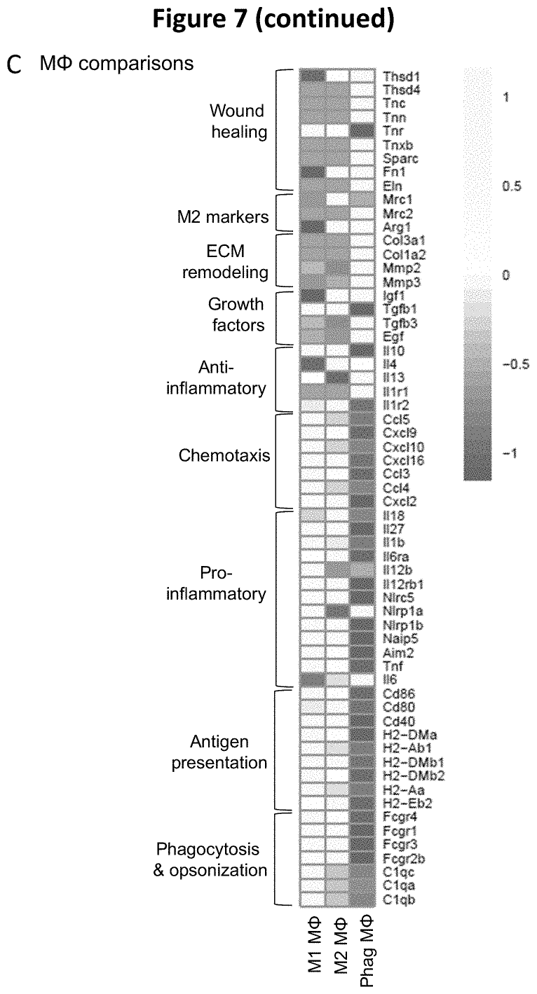

Using differential gene expression analysis, we first assessed the impact of our treatments on the M1-like and M2-like macrophage clusters in comparison to control (FIGS. 7A and 7B). Of note, these two macrophage clusters do not demonstrate evidence for hyper-phagocytosis of tumor cells at this time point of analysis, as evidenced by their lack of tumor marker uptake (FIG. 6B). Gene expression data revealed our treatments promoted macrophage polarization into a pro-inflammatory antitumor phenotype, as evidenced by an increase in genes involved in interferon, inflammatory cytokines, chemokines and TLR pathways (FIGS. 7A and 7B). Accordingly, these changes were the most significant with combination therapy, and also more strongly observed in the M1-like MΦ cluster.

In contrast, the Phag MΦ cluster (predominantly presence in the combination treatment group) have surprisingly increased expression of gene signatures for wound-repair (e.g. Thrombospondins and Tenascins), ECM remodeling (e.g. Collagens and MMPs), growth factors (e.g. Igf1, Tgfb and Egn and anti-inflammatory genes (e.g. IL4, IL13, IL1r) compared to the other two MΦ clusters (FIG. 7C). This is also accompanied by decreased expression of genes for pro-inflammatory cytokines/chemokines, phagocytosis/opsonization, and antigen presentation (FIG. 7C).

These scRNAseq analyses revealed that while Trastuzumab with CD47 blockade polarizes macrophages into an antitumor phenotype and greatly increases tumor phagocytosis, prolonged treatment and continuous tumor hyper-phagocytosis may also trigger a transcriptional switch in TAMs for repair of ADCP-induced tissue damage. Thus, while these studies demonstrate the antitumor efficacy of Trastuzumab+CD47 blockade, it also suggest that prolonging this process can trigger a wound healing response in macrophages that could have pro-tumor and/or immunosuppressive functions (44-46).

### Human CD47 Gene Expression is a Prognostic Factor in HER2+ Breast Cancer and Limits the Therapeutic Activity of Trastuzumab

As all of our investigations had been performed on different murine HER2+ BC models, we also wanted to determine if ADCP activity of Trastuzumab can be seen in human HER2+ BC and if CD47 could likewise limit its antitumor efficacy. Based on our findings, we hypothesized that CD47 expression may allow for resistance and reduced survival of HER2+ BC patients undergoing Trastuzumab therapies. To investigate this hypothesis, we utilized the METABRIC (Molecular Taxonomy of Breast Cancer

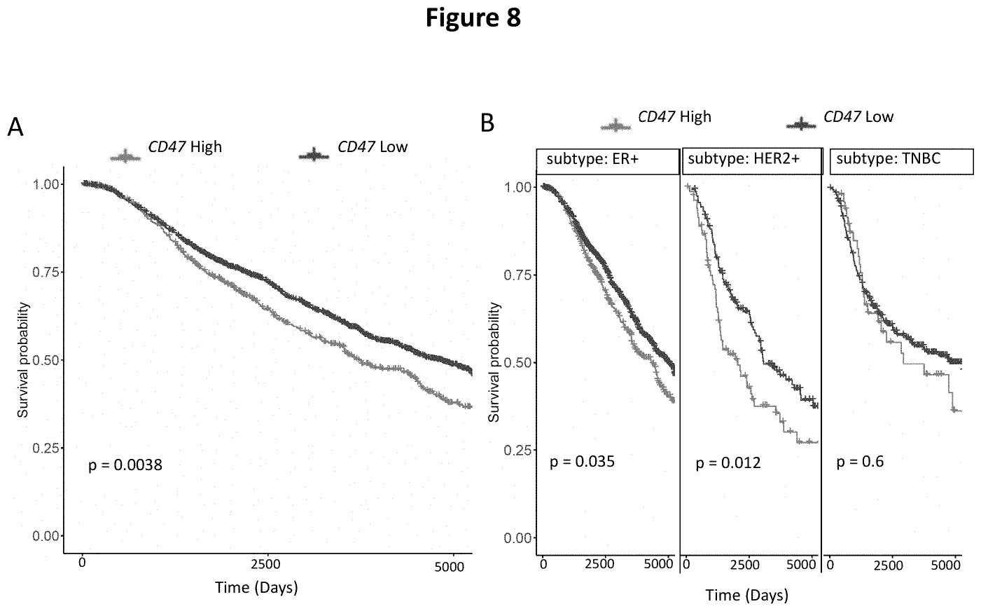

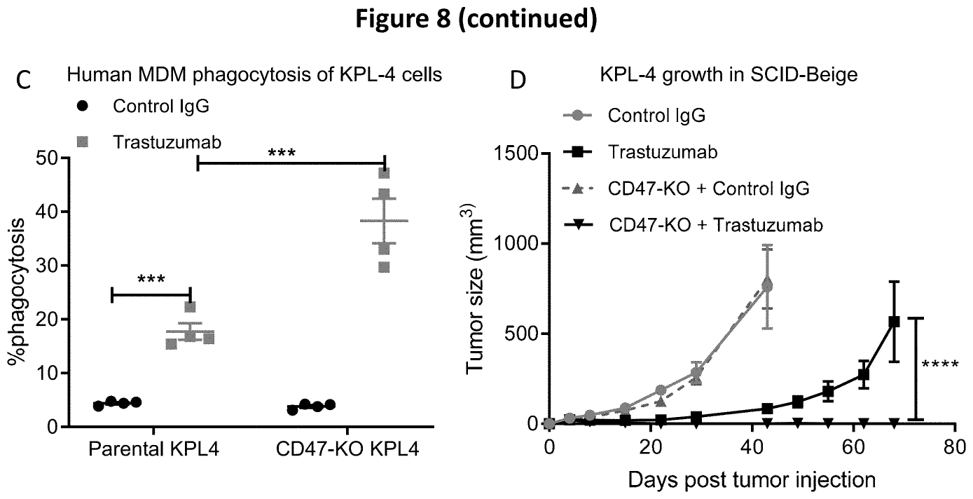

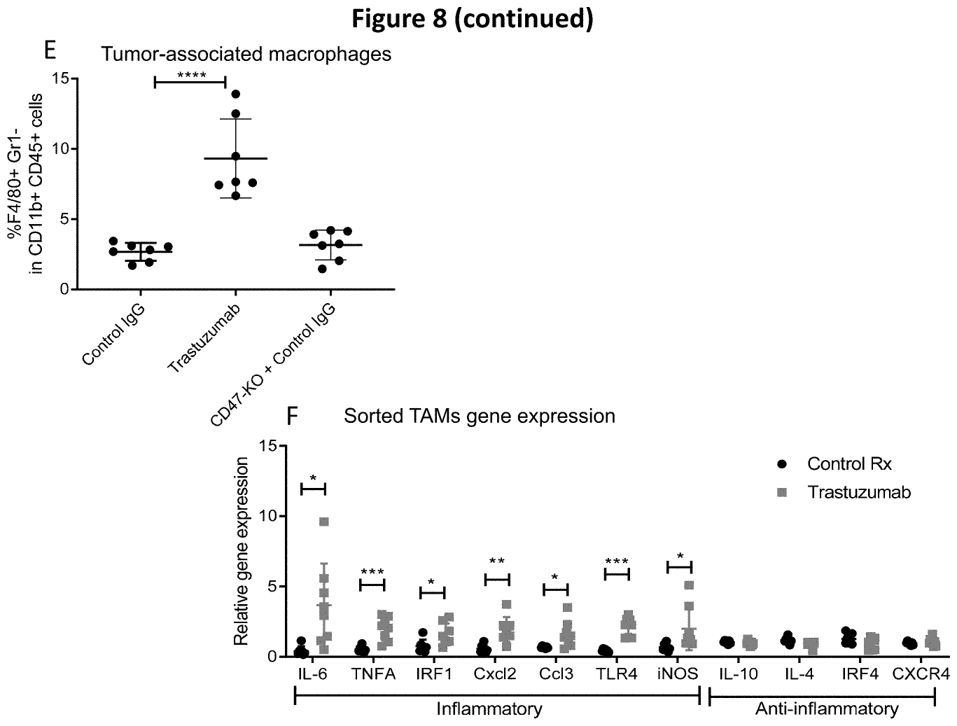

International Consortium) gene expression dataset (47) and stratified breast cancer patients of different molecular subtypes into “CD47 high” and “CD47 low” groups based on optimum threshold. This analysis revealed that CD47 gene expression associates with lower patient overall survival (FIG. 8A) and was most significant in the HER2+ molecular subtype compared to TNBC or ER+ subtypes (FIG. 8B). This suggests that CD47 signaling may be an important resistance mechanism for HER2+ breast cancer and Trastuzumab therapy.

We next investigated whether human CD47 limits the ADCP effect of Trastuzumab against amplified HER2+ human BC cells. To address this in vitro, we first generated CD47-KO KPL-4 (HER2+ BC) cells (FIG. 12C) and compared them to controls after Trastuzumab treatment in ADCP experiments using human PBMC derived macrophages. As in mouse studies, we found loss of human CD47 in tumor cells increased their susceptibility to ADCP elicited by Trastuzumab (FIG. 8C). To determine if this antitumor effect also occurs in vivo against human HER2+ BC cells, we implanted KPL-4 control and CD47-KO cell lines into SCID-beige mice (which contain a mouse SIRPα that can bind to human CD47 (48)) and treated with clinical grade Trastuzumab. As before, we saw a strong effect from Trastuzumab treatment that was significantly enhanced with CD47-KO, resulting in tumors being completely eliminated (FIG. 8D). In Trastuzumab-treated mice, we again found a significant increase of TAMs (FIG. 8E) and an upregulation of pro-inflammatory genes (FIG. 8F) as seen in the murine tumor model. Unfortunately, the complete regression of CD47KO+Trastuzumab tumors precluded any further analysis of these tumors. Collectively, these studies suggest that the dominant antitumor mechanism of Trastuzumab therapy is through ADCP of HER2+ tumor cells, which can be substantially impaired through the CD47-SIRPα axis. This suggests that combinatorial therapy with CD47 blockade could be beneficial in patients with Trastuzumab resistance.

### Discussion

Even since the demonstration of clinical benefit provided by therapeutic HER2 specific mAbs to patients with HER2 overexpressing BC, the mechanism of action for the therapeutic HER2 mAb, Trastuzumab has been the subject of numerous studies. Some reports suggest that Trastuzumab may both block oncogenic HER2 signaling as well as inducing ADCC (7, 49, 50). Using reflective murine versions of clinically approved HER2 specific mAb Trastuzumab, our in vitro studies confirmed these reported MOAs, specifically blockade of HER2 signaling and Trastuzumab-mediated ADCC by NK cells. In contrast, the in vivo antitumor mechanisms of Trastuzumab/4D5 remain less conclusive, with early studies suggesting the importance of signal blockade (51, 52), and subsequent studies demonstrating the direct involvement of ADCC eliciting FcR-expressing cells (10) (such as neutrophils and NK cells), and more recent studies highlighting the importance of adaptive immunity) (8, 15). Notably, few studies have examined Trastuzumab-mediated ADCP with a single study documenting the ability of Trastuzumab to elicit ADCP in vivo (16), while another study suggested that Trastuzumab-mediated ADCP from tumor-associated macrophages (TAMs) is immunosuppressive (17). Consequently, our novel models and agents provided a reliable platform and opportunity to interrogate the in vivo antitumor mechanism of HER2 specific mAbs against HER2 driven BC.

In this study using multiple models of human HER2 expressing BC, i.e. MM3MG-HER2Δ16, KPL-4 and an endogenous transgenic HER2+ BC model that is tolerant to human HER2, and using the murine version of Trastuzumab with the functionally equivalent mouse isotype (4D5-IgG2A), we demonstrate that macrophages are the major effectors carrying out the antitumor immunity of Trastuzumab therapy through antibody-dependent-cellular-phagocytosis (ADCP). Although TAMs have been shown to promote tumor progression, it is known that they also retain their Fc-dependent antitumor function when induced by targeted therapies (i.e. monoclonal antibodies) (53, 54). Our conclusion about the therapeutic impact of TAMs is supported by the following findings: (1) the therapeutic effect of Trastuzumab is equivalent in wild type and in SCID-beige mice and does not alter systemic HER2-specific adaptive immunity and T cell/NK cell infiltration in tumors, indicating adaptive immunity and NK cells are not necessary immune cells to mediate antitumor effects; (2) The depletion of macrophages but not neutrophils had a significant negative effect on Trastuzumab efficacy, (3) Trastuzumab treatment greatly and consistently increased TAMs frequency; (4) Trastuzumab treatment induced ADCP of HER2+ tumor cells in vitro and in vivo in a Fc-receptor dependent fashion; (5) Blocking of the innate immune ADCP CD47-SIRP1α regulatory axis significantly enhanced Trastuzumab therapeutic outcomes and also increased ADCP of tumor cells; (6) Trastuzumab combination with CD47 blockade induced TAMs into a highly phagocytic, immune-stimulatory and antitumor phenotype but also produced a wound-healing, immune-regulatory group of TAMs after prolonged tumor phagocytosis.

Our study provides insight on the potential of utilizing TAMs as a potent mediator of innate antitumor immunity that can be further exploited. It was initially believed that macrophages were present in high numbers in solid tumors as a mechanism of rejection. However, it soon became clear that TAMs are typically unable to induce an effective antitumor response in the immunosuppressive tumor microenvironment (55). Furthermore, high TAMs infiltration levels are often associated with poor patient prognosis in breast, lung, prostate, liver, thyroid, pancreas, kidney and many other solid cancer malignancies (56). Indeed, studies have shown that immunosuppressive TAMs can support tumor development by promoting angiogenesis, tissue invasion, metastasis and suppressing tumor attack by NK and CTL cells (57). In contrast, TAMs in colorectal cancer have a more activated, immune-stimulatory phenotype and interestingly, high TAM density in colorectal cancer correlates with increased patient survival, (54, 58). Nonetheless, TAMs in multiple histologic types of tumors retain their expression of Fcγ-receptors and increasing evidence suggests mAbs can phenotypically modify immunosuppressive TAMs towards an antitumor phenotype (53, 54, 59). As such, the manipulation of TAMs, potentially through a tumor targeting mAb (e.g. Trastuzumab) or targeting of regulatory axis receptors (e.g. CD47/SIRPα), are promising therapeutic approaches for multiple types of cancer.

While previous studies (8, 9) have documented the involvement of T cell immunity in mediating HER2 mAbs efficacy, we were unable to detect a significant enhancement of adaptive T cell responses with Trastuzumab monotherapy in either our orthotopic or HER2-tolerant endogenous models of HER2+ BC. This may be due to the nature of our tumor models, the timing of our analysis, or the specific mAb utilized. In our immunocompetent in vivo studies, we utilized both murine and human HER2 mAbs similar to Trastuzumab (isotypes with a high A/I ratio), as well as both human HER2 transformed cells and an endogenous mouse model of HER2+ BC. Previous studies (8, 9, 17) have utilized rat neu expressing ErbB2 models, non-HER2 transformed cells, and/or alternate Ab isotypes (mouse IgG1 with a low A/I ratio), which may account for a lack of ADCP activity and alteration of immunogenicity. Of note, a recent study using 4D5 antibody containing mouse IgG1 isotype reported that HER2 mAb elicited macrophage ADCP is an immunosuppressive mechanism (17). Given that the mouse IgG1 subclass strongly activates inhibitory FCGR signaling on effector cells (low A/I ratio) and therefore being very different from Trastuzumab (human IgG1, high A/I ratio) (18, 19, 40), this emphasizes the need of using functionally equivalent mouse isotypes in translational studies to accurately model human antibody therapy. Nevertheless, clinical studies have demonstrated significant associations between adaptive immune responses and Trastuzumab+chemotherapy efficacy (60). Phagocytosis of tumor cells by macrophages has been documented to boost the priming of tumor specific adaptive CD4+ and CD8+ T cells (36, 61), while different types of chemotherapy have been documented to enhance phagocytosis and augment immunogenic tumor cell death (62). Taken together, we believe that the clinical use of immunogenic chemotherapy combinations could stimulate adaptive immunity that would be potentially enhanced by Trastuzumab-mediated ADCP. However, in the absence of strong immune-stimulation (potentially through chemotherapy or immunogenic cell lines), Trastuzumab does not appear highly effective at eliciting adaptive immunity and functions mainly through the stimulation of ADCP.

In identifying ADCP as a critical mechanism for Trastuzumab efficacy, we also explored if it could be further enhanced through the blockade of the CD47 innate immune checkpoint. CD47 is highly expressed in BC and functions to suppress phagocytosis through binding with SIRPα on macrophages (23, 24). Interestingly, we found CD47 gene expression is a negative prognostic factor in human BC, most significantly in HER2+ BC. As treatment of HER2 overexpressing tumors with Trastuzumab has been available for many years, this observation suggests that CD47 may be functioning in Trastuzumab-treated patients to mediate ADCP/therapeutic resistance. This conclusion is supported by the enhanced effects observed between Trastuzumab and CD47 blockade in augmenting ADCP and antitumor effects in our study. Moreover, single cell transcriptome analysis of the tumor microenvironment demonstrates that Trastuzumab therapy stimulates TAMs into a pro-inflammatory antitumor phenotype, which is further boosted by CD47 blockade (FIGS. 7A and 7B). Such changes in macrophage phenotypes were also observed in co-cultured ADCP experiments. This suggests combination of targeted mAbs therapy with CD47-SIRPα blockade could be beneficial in HER2+ BC and potentially other solid tumors. Proof-of-concept studies using tumor-targeting mAbs and CD47 blockade have been demonstrated in preclinical lymphoma models, as well as a recent phase I study of anti-CD20 mAbs (Rituximab) and CD47 blockade, in Rituximab-refractory Non-Hodgkins Lymphoma patients (41, 63).

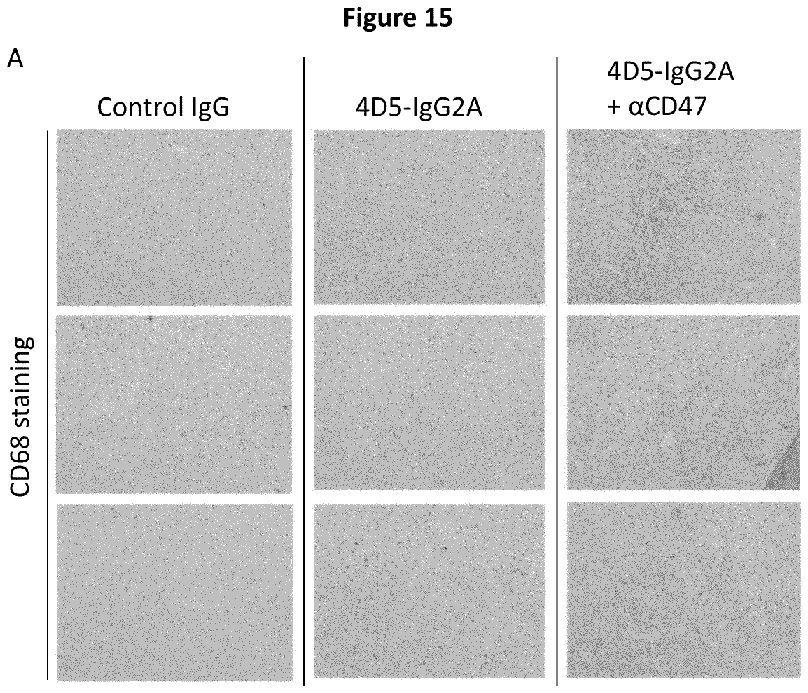

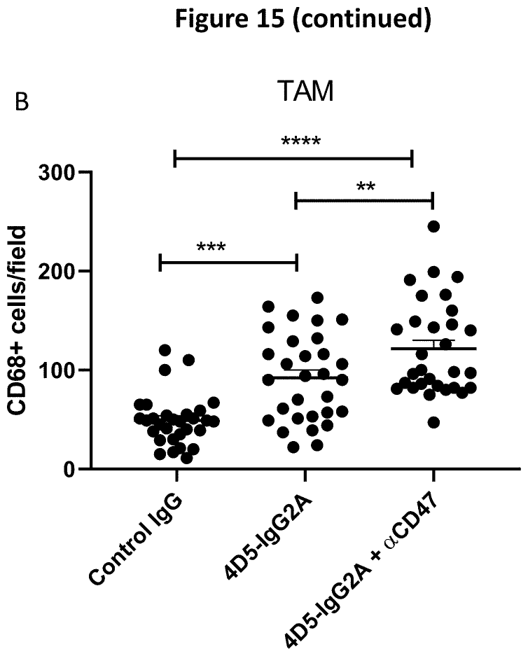

Additionally by implementing different methods, such as multi-color FACS analysis and single-cell transcriptome analysis, we are the first to demonstrate in vivo tumor phagocytosis by macrophages upon combination of Trastuzumab with CD47 blockade therapies. Moreover, we were able to identify a distinct cluster of hyper-phagocytic TAMs within the TME. The identification of this population of TAMs may also serve as a predictive biomarker of this form of therapy. Gene expression analysis suggested that after profound phagocytosis of tumor cells, these macrophages switched to a tissue repair phenotype, as evidenced by their upregulation of gene signatures for wound-healing, growth factors, ECM remodeling, and anti-inflammatory markers compared to resident macrophages (FIG. 7C). Indeed, several studies have demonstrated that cellular phagocytosis over time influences macrophage phenotype, causing a switch from pro-inflammatory to a growth promoting, reparative phenotype (44-46). Interestingly, while the total number of CD8+ TILs were reduced by prolonged combination therapy, the relative percentage of cytotoxic T cells was greatly increased (FIG. 15), possibly suggesting a boost in overall tumor-specific T cells frequency. In this manner, this combination therapy may allow for enhanced tumor antigen presentation at the earlier time points of treatment through increasing tumor phagocytosis and antigen uptake, while prolonged treatment limits general T cell infiltration after progression to a wound-healing TAM phenotype. Future experiments using Trastuzumab+αCD47 mAbs analyzing multiple treatment time points, reducing the length of treatment, or combining with other immune checkpoint blockades could potentially improve the infiltration of tumor-specific CTLs.

While this is an area in need of additional study, our results suggest that strategies to specifically enhance ADCP activity may be critical in overcoming resistance to HER2 mAb therapies by inhibiting tumor growth and potentially enhancing antigen presentation. While only a single clinical trial using combination of a therapeutic mAb (anti-CD20) and CD47 mAbs has been reported, this study demonstrated a ˜50% response rate (11 of 22 patients) and ˜36% complete response rate (8 of 22 patients) in resistant/refractory non-Hodgkins' lymphoma (41). These clinical findings, in conjunction with our recent preclinical studies, strongly suggest combination therapy approach of Trastuzumab with CD47-SIPRα checkpoint blockade could potentially show more benefits and insights of Trastuzumab therapy in HER2+ BC patients. However, the transcriptional switch seen in macrophages after prolonged ADCP also requires attention in future studies that utilize CD47 blockade in combination with targeted mAbs.

In sum, our study suggests that the dominant therapeutic MOA for Trastuzumab is through its elicitation of TAM mediated ADCP, which can be enhanced by strategies to specifically augment ADCP. This has potential implications for the use of Trastuzumab in HER2+ cancers, as well as the utilization of other targeted therapies (such as EGFR, CD20, etc.), where efforts to enhance and control ADCP have not been prioritized.

### Methods

### Cell Lines and Genetic Modifications Strategies

Mouse mammary gland cell lines MM3MG and EPH4 were obtained from ATCC and cultured as described by ATCC protocol. The cDNA of a naturally occurring splice variant of human HER2 (HER2Δ16),), or wild type HER2, were transduced into MM3MG and NMUMG cells using lentiviral transduction. Human HER2+ breast cancer cell line KPL4 was a kind gift from Dr. Kurebayashi (University of Kawasaki Medical School, Kurashiki, Japan) (64) and SKBR3 were purchased from ATCC and cultured as described by ATCC protocol. Jurkat-NFAT-LUC line were obtained from Invivogen (jktl-nfat). CRISPR-Cas9 approached were used to knockout mouse Cd47 in MM3MG-HER2Δ16 cells or human CD47 in KPL4 cells. Gene targeting of mouse Cd47, human CD47 and control gene GFP by CRISPR/Cas9 was accomplished through the use of pLentiCRISPRv2 (Addgene plasmid #52961) using published protocols (65). Genes were targeted using the guide sequences (CCCTTGCATCGTCCGTAATG (SEQ ID NO: 6) and GGATAAGCGCGATGCCATGG (SEQ ID NO: 7)) for mouse Cd47, (ATCGAGCTAAAATATCGTGT (SEQ ID NO: 8) and CTACTGAAGTATACGTAAAG (SEQ ID NO: 9)) for human CD47, and (GGGCGAGGAGCTGTTCACCG (SEQ ID NO: 10)) for the GFP control. Successful targeting of CD47 was determined by flow cytometry screening after single cell clonal selection. The overexpression vector of mouse Cd47 was generated by synthesizing the Cd47 gene and cloning it into pENTR1a (using NEB Gibson Isothermal Assembly Mix) and then using L/R clonase to generate expression lentiviruses (pLenti-CMV-Puro) and cells were selected using puromycin.

### Mice

Female Balb/c (Jackson Labs, Bar Harbor, MA), SCID-beige (C.B-Igh-1b/GbmsTac-Prkdc^{scid}-Lystbg N7; Taconic Biosciences, Model# CBSCBG), Fcer1g^{-/-} (C.129P2(B6)-Fcer1g^{tm1Rav} N12; Taconic Biosciences, Model#584) mice between the ages of 6 and 10 weeks old were used for all experiments. The HER2Δ16 transgenic model was generated by crossing MMTV-rtTA strain (a kind gift by Dr. Lewis Chodosh, UPenn, Philadelphia, USA) with TetO-HER2d16-1RES-EGFP strain (a kind gift by Dr. William Muller, McGill University, Montreal, Canada) (20). 6-weeks old mice were put on doxycycline diet and enrolled for experiments when they develop palpable breast tumor (usually in 4-6 weeks post dox diet).

### Therapeutic Antibodies and Other Experimental Reagents

Clinical Grade Trastuzumab (human IgG1) were obtained from Duke Medical Center. 4D5, the murine version of Trastuzumab (with the IgG2A and IgG1 mouse isotypes) were produced by GenScript through special request. CD47 Blockade antibody MIAP410 (BE0283) and control mouse IgG2A (BE0085) were purchased from BIOXCELL. Neutrophil depletion anti-LY6G antibody (IA8, BP0075-1) and macrophage depletion antibody anti-CSF1R (AS598, BE0213) were purchased from BIOXCELL. Clodronate liposomes were purchased from www.clodronateliposomes.org

### Orthotopic Implanted HER2+ Breast Cancer Mouse Models and Therapeutic Antibody Treatments

MIVI3MG cells expressing human HER2Δ16 were implanted into their mammary fat pads (1×10⁶ cells) of Balb/c mice. For the human xenograft model, KPL-4 cells (1×10⁶ cells) were implanted into mammary fat pads (A/FP) of SCID-Beige Balb/c mice. Tumor growth were measured with caliper-based tumor volume measurement (length×width×depth) over time. For therapeutic treatments, Trastuzumab or 4D5 were administered weekly (200 μg per mice intraperitoneally) around 4-5 days post tumor implantation. CD47 blockade (MIAP410) were administered weekly when indicated (300 μg per mice intraperitoneally) around 4-5 weeks post tumor implantation. For macrophage depletion, anti-CSF1R antibody were administered triweekly (300 μg per mice intraperitoneally), starting at two weeks before tumor implantation and with treatment maintained over the course of the experiment. Clodronate liposomes were administered biweekly (100 μL per mice, intraperitoneally). For neutrophil depletion, anti-LY6G antibody were administered biweekly (300 μg per mice intraperitoneally) for the first two weeks post tumor implantation.

### Transgenic HER2Δ16 Mouse Model and Therapeutic Antibody Treatments

The HER2Δ16 transgenic mouse model was generated by crossing two strains of mice, TetO-HER2Δ16-1RES-EGFP and MJI/TV-rtTA. This system was described previously (20), but utilizes a TET-ON system (with MTV-rtTA) to drive expression of HER2Δ16 to generate HER2+ BC. For experiments, one-month old mice were put on Doxycycline diet (200 mg/kg, Bio-Serv, Flemington, N.J.) to induce spontaneous HER2-driven breast cancer. Individual animals were randomly enrolled into a specific treatment group as soon as palpable breast tumors were detected (˜200 mm³) in any of the eight mammary fat pads. Control and 4D5-IgG2A antibodies were treated 200 μg weekly, whereas MIAP410 were treated 300 μg weekly intraperitoneally. Animals were terminated once their total tumor volume reached >2000 mm³.

### Flow Cytometry Analysis of Tumor Infiltrating Immune Cells

When tumor growth reached humane end point size (>1000 mm³), whole tumors from mice were harvested and cut into <1 mm small pieces, and incubated for 1 hour in digestion buffer (DMEM+100 μg/mL collagenase+0.2 U/mL DNAse+1 μg/mL hyalurodinase). Single cell suspensions were spin down through a 70 μm filter and washed with medium. Approximately 5 million cells were used for staining and flow cytometry analysis. The following panel of immune cell markers (Biolegend) were used: CD45 BV650, CD11b PE-Cy7, LY6G APC, LY6C BV410, F4/80 PerCP-CY5.5, CD8B APC-CY7, CD4 PE-TR, CD49b FITC and viability dye (Aqua or Red). Tumor-associated macrophages (TAM) were identified by F4/80+ LY6G− LY6C− CD11b+ CD45+ gating. LY6G+ neutrophils were identified by LY6G+ CD11b+ CD45+ gating, whereas LY6C+ monocytes were gated on LY6C+ CD11b+ CD45+ cells.

### In Vivo ADCP Assay

MM3MG-HER2Δ16 cells were labeled with Vybrant DiD labeling solution (Thermo V22887) according to manufacturer's protocol, and labeled cells were implanted (1×10⁶) into MFP of Balb/c mice. Once tumor reaches around 1000 mm³ in sizes, mice were treated with either control antibody (200 μg), 4D5 (200 μg), or 4D5 in combination with MIAP410 (300 μg) per day for two consecutive days. Tumor associated macrophages were analyzed by FACS (CD11b+, F4/80+, LY6G−, LY6C−) and the percentage of TAMs that have taken up DiD-labeled tumor cells were quantified for in vivo ADCP analysis.

### In Vitro ADCP and ADCC Assays

ADCP and ADCC by macrophages—Bone marrow derived macrophages (BMDM) were generated from mouse tibia, differentiated for 10 days with 50 ng/mL mouse MCSF (Peprotech 315-02). Briefly, 50 million bone marrow cells were plated in 10 cm² tissue culture dish with MCSF on day 0. Unattached cells in supernatant were removed and fresh media+MC SF were added on day 3, day 6 and day 9. BMDM were used for ADCP/ADCC assays on day 10. Tumor cells MM3MG-HER2416 were labeled with Brilliant Violet 450 Dye (BD 562158) according to manufacturer's protocol, and incubated with control or anti-HER2 antibodies (10 μg/mL) in 96-wells (100,000 cells/well) for 30 minutes at 37° C. BMDM were then added for co-culture at a 3:1 ratio of Tumor vs BMDM. After 2 hours co-culture, phagocytosis of BV450-labeled tumor cells by BMDM were analyzed by FACS with CD45-APC staining and Live-death (Red) staining. When indicated, ADCP inhibitor Latrunculin A (120 nM, Thermo L12370) and ADCC inhibitor Concanamycin A (1 μM, Sigma C9705) were added as assay controls. For human macrophages ADCP assay, human monocytes-derived macrophages (hMDM) were generated from three donors' PBMCs. hMDM were generated with 50 ng/mL human MCSF (Peprotech 300-25) and 50 ng/mL human GM-CSF (Peprotech 300-03). KPL-4 cells were used as human HER2+ tumor targets and labeled and co-cultured similarly as with mouse ADCP assay.

### FCGR Binding/Activation Assay

Jurkat cells expressing mouse Fcgr1, Fcgr2b, Fcgr3 or Fcgr4 with NFAT-Luciferase reporter were generated with lentiviral transduction and selected with puromycin (validated in FIG. 12D-F). For the assay, MM3MG breast cancer lines expressing HER2 were first plated and treated with Trastuzumab or 4D5 antibodies or control IgG for 1 hour. Jurkat-FCGR-NFAT-LUC effector cells were added and co-cultured for 4 hours. FCGR signaling activation were assessed by luciferase activity quantification.

### Multiplex Cytokine and Chemokine Assay

BMDM were co-cultured with MM3MG-HER2Δ16 cells for 24 hours, and supernatants were harvested for analysis of cytokines/chemokines levels. The 26-Plex Mouse ProcartaPlex™ Panel1 kit (Thermo) was used and analyzed using the Luminex MAGPIX system.

### METABRIC Analysis of CD47 Expression in Breast Cancer Patients

Pre-processing METABRIC data: Previously normalized gene expression and clinical data were obtained from the European Genome-Phenome Archive (EGA) under the accession id EGAS00000000098 after appropriate permissions from the authors (47). The discovery dataset was composed of 997 primary breast tumors and a second validation set was composed of 995 primary breast tumors. The expression data were arrayed on Illumina HT12 Bead Chip composed of 48,803 transcripts. Multiple exon-level probe sets from a transcript cluster grouping were aggregated to a single gene-level probe set using maximum values across all the probes for a given gene. The resulting gene expression matrix consists of 28,503 genes.
In order to assess the prognostic significance of CD47 in METABRIC data we generated Kaplan-Meier survival curves on patients stratified by the average expression of CD47 (in to low and high groups) using R package ‘survminer’ (version 0.4.3). Distributions of Kaplan-Meier survival curves for progression-free and overall survival were compared using log-rank test, and a log-rank test p-value ≤0.05 is considered to be statistically significant.

### Single-Cell RNA-Seq Analysis

Fastq files from 10× library sequencing were processed using the CellRanger pipeline available from 10× genomics. As part of the processing the assembled sequencing reads were mapped to the mm10 mouse genome. In order to obtain the transcript counts of human ERBB2 (HER2) the sequencing reads were separately aligned to the current version of the human genome, GRCh38.

The gene expression files consisting of raw counts at the gene level for each cell which was analyzed using version 2.3.4 of the Seurat package. The human ERBB2 counts were combined with the mm10 based counts into once expression matrix for each sample. Briefly, the data analysis steps using Seurat consisted of combining the gene counts for all the cells in the different conditions into one matrix, filtering low quality cells, normalizing, and adjusting for cell cycle and batch effects. Unsupervised clustering was done to separate the cell types and markers for the cell types were identified using differential gene expression. These markers were then used for identifying the cell subpopulations within the tumor microenvironment, namely the Immune cells, Tumor cells and Fibroblasts. The normalized gene counts were used to generate tSNE maps for visualization of the cell types and heatmaps for the cell type specific gene expression. Expression of predefined gene sets representing pathways of interest where obtained from previous publications and summarized in Table S2 (FIG. 16). The data discussed in this publication have been deposited in NCBI's Gene Expression Omnibus and are accessible through GEO Series accession number GSE139492 (ncbi.nlm.nih.gov/geo/query/acc.cgi?acc=GSE139492)

### Statistical Methods

All statistical analysis of tumor growth comparisons and tumor immune infiltrates were performed with GraphPad Prism (v8) using two-way ANOVA or one-way ANOVA test with Tukey's multiple comparisons. Unless otherwise indicated in the figure, tests results were shown between treatment vs control group. Group sizes for animal tumor growth experiments were determined based on preliminary datasets. All subjects in animal experiments were randomized into a treatment or control group. For in vitro experiments, i.e. ADCP/ADCC/CDC assays, ELISPOT assays, FCGR signaling assay and cytokine assays, all data were statistically analyzed by one-way ANOVA test with Tukey's multiple comparisons, and performed with at least four biological replicates per experiment and repeated at least two times. RT-qPCR data were analyzed by two-sided Student's t test for each target gene. 95% confidence interval was considered for statistics and p<0.05 was considered significant.

### RT-qPCR Analysis of Sorted Macrophages

KPL4 xenografts were processed into single cell suspensions as described above, and tumor associated macrophages were sorted by FACS (Live CD45+ CD11b+ Gr1− and F4/80+). RNA were isolated from sorted macrophages using RNeasy Mini Kit (Qiagen) and cDNA were generated using “All-in-One cDNA Synthesis Supermix (Biotool B24403). RT-qPCR were performed using 2× SYBR Green qPCR Master Mix (Biotool B21202).

### In Vitro CDC Assay

Complement-dependent cytotoxicity (CDC) assay—MM3MG-HER2Δ16 or MM3MG cells expressing luciferase were incubated with 2 μg/mL of anti-HER2 antibodies for 1 hour at 37° C. After incubation, human or rabbit serum (non heat-inactivated) were added to culture to a final concentration of 25% serum. After 4 hours, cells were lysed and viability were assessed by luciferase expression. Heat inactivated serum was used as negative control. A combination of different HER2-targeting antibodies were used as positive control, as this will greatly increase antibody-mediated CDC activity (unpublished results).

### HER2 Signaling Assays

HEK 293T cells stably expressing doxycycline-inducible HER2Δ16 were transfected (lipofectamine 2000) with luciferase reporter constructs (5 μg g of DNA in 2×10\hat{ }6 cells) for MAPK/ERK or AP-1/c-JUN pathways activation. Reporter constructs were originated from Cignal Reporter Assay Kit (336841, Qiagen). 12 hours after transfection and dox treatment, cells were treated with of 4D5 or Trastuzumab or lapatinib (Kinase inhibitor of HER2 signaling as assay positive control) at the concentrations as indicated in the results. HER2 signaling activity were analyzed by luciferase readout of MAPK/ERK and AP-1/c-JUN pathway reporters. Non-induced (no dox treatment) cells were used as negative control.

### ELISPOT Assay

Mouse splenocytes were harvested by mashing whole spleens into single cells through a 40 μm filter. Red blood cells were lysed for 15 minutes using RBC lysis buffer (Sigma R7757). Live Splenocytes were then counted using the Muse® Cell Analyzer. For adaptive T cell response analysis, we used the mouse IFN-γ ELISPOT (MABTECH 3321-2H) with manufacturer's protocol. Briefly, 500,000 splenocytes were incubated in RPMI-1640 medium (Invitrogen) with 10% fetal bovine serum for 24 hours with peptides at a final concentration of 1 μg/mL. For HER2-specific responses, 169 peptides spanning the extracellular domain of HER2 protein were used. We used irrelevant HIV-1 Gag peptides (1 μg/mL, JPT, Germany) as control peptides. PMA (50 ng/ml) and Ionomycin (1 μg/ml) (Sigma) were used as positive controls.

### Library Preparation for Single Cell RNA-Seq

Tumors from treated transgenic mice were harvested and processed into single cell suspension using Mouse Tumor Dissociation Kit (Miltenyi, 130-096-730) following manufacturer's protocol with recommendations for 10× Genomics platform use (10× genomic manual, CG000147). Single cell suspensions from tumors were treated with red blood cells lysing buffer (Sigma R7757) for 5 minutes, and stained with “Fixable Far Red Dead Cell Stain Kit” (L10120). Live singlets (single cells) from tumor suspension were sorted by FACS and counted using hemocytometer. To generate 10× Genomics libraries, we used Chromium Single Cell 5′ Library Construction Kit (PN-1000020) following manufacturer's protocol. A targeted cell recovery of 4000 cells was used for each tumor sample. Generated cDNA libraries were quality checked on Agilent Bioanalyzer 2100 and submitted to MedGenome Inc for sequencing on NovaSeq S4 instrument.

### Immunohistochemistry (IHC) Staining of TAMs

Tumor tissues (˜3 mm³) were fixed in 4% PFA overnight at 4° C. and then paraffin-embedded. Tumor sections in vertical slide holder were deparaffinized with two xylene washes and hydrated by graded ethanol washes (100%, 95%, 80%, 70%). Antigens were unmasked by heat treatment in 10 mM sodium citrate buffer (pH 6.0) for 15 minutes. Endogenous peroxidase activity were quenched in 30% peroxide for 15 minutes. Blocking of non-specific antigen bindings were performed by incubation in 5% BSA 30 minutes. Primary antibody incubation (anti-CD68, Abcam ab125212) overnight at 4° C. After wash, stained antigens were detected using SignalStain Boost IHC Detection Reagent (HRP, Rabbit) from (Cell Signaling, 8114) according to manufacturer's protocol. Substrate development were performed using DAB Peroxidase Substrate Kit (Vector Lab, SK-4100) for about 2 minutes. Slides were then counterstained in hematoxylin solutions, dehydrated through graded ethanol washes, cleared with two xylene washes, and covered with mounting medium. CD68+ staining were quantified in five random fields per slide at 20× magnification, and the average counts was reported.

### REFERENCES

1. Slamon D J, Clark G M, Wong S G, Levin W J, Ullrich A, and McGuire W L. Human breast cancer: correlation of relapse and survival with amplification of the HER-2/neu oncogene. Science (New York, N.Y.). 1987;235(4785):177-82.
2. Di Fiore P P, Pierce J H, Kraus M H, Segatto O, King C R, and Aaronson S A. erbB-2 is a potent oncogene when overexpressed in NIH/3T3 cells. Science (New York, N.Y.). 1987;237(4811):178-82.
3. Hudis C A. Trastuzumab—mechanism of action and use in clinical practice. The New England journal of medicine. 2007;357(1):39-51.
4. Untch M, Rezai M, Loibl S, Fasching P A, Huober J, Tesch H, et al. Neoadjuvant treatment with trastuzumab in HER2-positive breast cancer: results from the GeparQuattro study. Journal of clinical oncology: official journal of the American Society of Clinical Oncology. 2010;28(12):2024-31.
5. Gianni L, Pienkowski T, Im Y -H, Tseng L -M, Liu M -C, Lluch A, et al. 5-year analysis of neoadjuvant pertuzumab and trastuzumab in patients with locally advanced, inflammatory, or early-stage HER2-positive breast cancer (NeoSphere): a multicentre, open-label, phase 2 randomised trial. The Lancet Oncology. 2016;17(6):791-800.
6. de Azambuj a E, Holmes A P, Piccart-Gebhart M, Holmes E, Di Cosimo S, Swaby R F, et al. Lapatinib with trastuzumab for HER2-positive early breast cancer (NeoALTTO): survival outcomes of a randomised, open-label, multicentre, phase 3 trial and their association with pathological complete response. The Lancet Oncology. 2014;15(10):1137-46.
7. Bianchini G, and Gianni L. The immune system and response to HER2-targeted treatment in breast cancer. The Lancet Oncology. 2014;15(2):e58-e68.
8. Park S, Jiang Z, Mortenson E D, Deng L, Radkevich-brown O, Yang X, et al. The Therapeutic Effect of Anti-HER2/neu Antibody Depends on Both Innate and Adaptive Immunity. Cancer Cell. 2010;18(2):160-70.
9. Stagg J, Loi S, Divisekera U, Foong S, Duret H, and Yagita H. Anti-ErbB-2 mAb therapy requires type I and II interferons and synergizes with anti-PD-1 or anti-CD137 mAb therapy. 2011:10-5.
10. Clynes R A, Towers T L, Presta L G, and Ravetch J V. Inhibitory Fc receptors modulate in vivo cytoxicity against tumor targets. Nature Medicine. 2000;6:443.
11. Musolino A, Naldi N, Dieci M V, Zanoni D, Rimanti A, Boggiani D, et al. Immunoglobulin G fragment C receptor polymorphisms and efficacy of preoperative chemotherapy plus trastuzumab and lapatinib in HER2-positive breast cancer. The pharmacogenomics journal. 2016;16(5):472-7.
12. Muntasell A, Cabo M, Servitja S, Tusquets I, Martinez-Garcia M, Rovira A, et al. Interplay between Natural Killer Cells and Anti-HER2 Antibodies: Perspectives for Breast Cancer Immunotherapy. Front Immunol. 2017;8:1544.
13. Muntasell A, Rojo F, Servitja S, Rubio-Perez C, Cabo M, Tamborero D, et al. NK Cell Infiltrates and HLA Class I Expression in Primary HER2(+) Breast Cancer Predict and Uncouple Pathological Response and Disease-free Survival. Clinical cancer research: an official journal of the American Association for Cancer Research. 2019;25(5):1535-45.
14. Albanesi M, Mancardi D A, Jonsson F, Iannascoli B, Fiette L, Di Santo J P, et al. Neutrophils mediate antibody-induced antitumor effects in mice. Blood. 2013;122(18):3160-4.
15. Mortenson E D, Park S, Jiang Z, Wang S, and Fu Y X. Effective anti-neu-initiated antitumor responses require the complex role of CD4+ T cells. Clinical cancer research: an official journal of the American Association for Cancer Research. 2013;19(6):1476-86.
16. Shi Y, Fan X, Deng H, Brerski R J, Rycyzyn M, Jordan R E, et al. Trastuzumab triggers phagocytic killing of high HER2 cancer cells in vitro and in vivo by interaction with Fcgamma receptors on macrophages. J Immunol. 2015;194(9):4379-86.
17. Su S, Zhao J, Xing Y, Zhang X, Liu J, Ouyang Q, et al. Immune Checkpoint Inhibition Overcomes ADCP-Induced Immunosuppression by Macrophages. Cell. 2018;175(2):442-57 e23.
18. Bruhns P, and Jonsson F. Mouse and human FcR effector functions. Immunol Rev. 2015;268(1):25-51.
19. Nimmerjahn F, and Ravetch J V. Divergent Immunoglobulin G Subclass Activity Through Selective Fc Receptor Binding. Science. 2005;310(5753):1510.
20. Turpin J, Ling C, Crosby E J, Hartman Z C, Simond A M, Chodosh L A, et al. The ErbB2ΔEx16 splice variant is a major oncogenic driver in breast cancer that promotes a pro-metastatic tumor microenvironment. Oncogene. 2016;35(47):6053-64.
21. Crosby E J, Wei J, Yang X Y, Lei G, Wang T, Liu C X, et al. Complimentary mechanisms of dual checkpoint blockade expand unique T-cell repertoires and activate adaptive anti-tumor immunity in triple-negative breast tumors. Oncoimmunology. 2018;7(5):e1421891.
22. Jaiswal S, Chao M P, Majeti R, and Weissman I L. Macrophages as mediators of tumor immunosurveillance. Trends in immunology. 2010;31(6):212-9.
23. Mantovani A, and Longo D L. Macrophage Checkpoint Blockade in Cancer—Back to the Future. New England Journal of Medicine. 2018;379(18):1777-9.
24. Huang Y, Ma Y, Gao P, and Yao Z. Targeting CD47: The achievements and concerns of current studies on cancer immunotherapy. Journal of Thoracic Disease. 2017;9(2):E168-E74.
25. Zhang H, Lu H, Xiang L, Bullen J W, Zhang C, Samanta D, et al. HIF-1 regulates CD47 expression in breast cancer cells to promote evasion of phagocytosis and maintenance of cancer stem cells. Proceedings of the National Academy of Sciences. 2015;112(45):E6215.
26. Yan M, Schwaederle M, Arguello D, Millis S Z, Gatalica Z, and Kurzrock R. HER2 expression status in diverse cancers: review of results from 37,992 patients. Cancer metastasis reviews. 2015;34(1):157-64.
27. Carter P, Presta L, Gorman C M, Ridgway JB, Henner D, Wong W L, et al. Humanization of an anti-p185HER2 antibody for human cancer therapy. Proceedings of the National Academy of Sciences. 1992;89(10):4285.
28. Stewart R, Hammond S A, Oberst M, and Wilkinson R W. The role of Fc gamma receptors in the activity of immunomodulatory antibodies for cancer. Journal for ImmunoTherapy of Cancer. 2014;2(1):1-10.
29. Moasser M M. Two dimensions in targeting HER2. Journal of clinical oncology: official journal of the American Society of Clinical Oncology. 2014;32(19):2074-7.
30. Moasser M M, and Krop I E. The Evolving Landscape of HER2 Targeting in Breast Cancer. JAMA Oncol. 2015;1(8):1154-61.
31. Wang Y, Yang Y J, Wang Z, Liao J, Liu M, Zhong X R, et al. CD55 and CD59 expression protects HER2-overexpressing breast cancer cells from trastuzumab-induced complement-dependent cytotoxicity. Oncol Lett. 2017;14(3):2961-9.
32. Gordon S R, Maute R L, Dulken B W, Hutter G, George B M, McCracken M N, et al. PD-1 expression by tumour-associated macrophages inhibits phagocytosis and tumour immunity. Nature. 2017;545(7655):495-9.
33. Willingham S B, Volkmer J P, Gentles A J, Sahoo D, Dalerba P, Mitra S S, et al. The CD47-signal regulatory protein alpha (SIRPa) interaction is a therapeutic target for human solid tumors. Proceedings of the National Academy of Sciences. 2012;109(17):6662-7.
34. Oliveira C A, Kashman Y Fau—Mantovani B, and Mantovani B. Effects of latrunculin A on immunological phagocytosis and macrophage spreading-associated changes in the F-actin/G-actin content of the cells. (0009-2797 (Print)).
35. Toda H, Araki K, Moritomo T, and Nakanishi T. Perforin-dependent cytotoxic mechanism in killing by CD8 positive T cells in ginbuna crucian carp, Carassius auratus langsdorfii. Developmental & Comparative Immunology. 2011;35(1):88-93.
36. Guilliams M, Bruhns P, Saeys Y, Hammad H, and Lambrecht B N. The function of Fcgamma receptors in dendritic cells and macrophages. Nat Rev Immunol. 2014;14(2):94-108.
37. Bournazos S, Wang T T, and Ravetch J V. The Role and Function of Fcgamma Receptors on Myeloid Cells. Microbiol Spectr. 2016;4(6).
38. Nimmerjahn F, Lux A, Albert H, Woigk M, Lehmann C, Dudziak D, et al. FcγRIV deletion reveals its central role for IgG2a and IgG2b activity in vivo. Proceedings of the National Academy of Sciences. 2010;107(45):19396.
39. Parekh B S, Berger E, Sibley S, Cahya S, Xiao L, LaCerte M A, et al. Development and validation of an antibody-dependent cell-mediated cytotoxicity-reporter gene assay. MAbs. 2012;4(3):310-8.
40. Dekkers G, Bentlage A E H, Stegmann T C, Howie H L, Lissenberg-Thunnissen S, Zimring J, et al. Affinity of human IgG subclasses to mouse Fc gamma receptors. MAbs. 2017;9(5):767-73.
41. Advani R, Flinn I, Popplewell L, Forero A, Bartlett N L, Ghosh N, et al. CD47 Blockade by Hu5F9-G4 and Rituximab in Non-Hodgkin's Lymphoma. N Engl J Med. 2018;379(18):1711-21.
42. Lawson M A, McDonald M M, Kovacic N, Hua Khoo W, Terry R L, Down J, et al. Osteoclasts control reactivation of dormant myeloma cells by remodelling the endosteal niche. Nature Communications. 2015;6:8983.
43. Turpin J, Ling C, Crosby E J, Hartman Z C, Simond A M, Chodosh L A, et al. The ErbB2DeltaEx16 splice variant is a major oncogenic driver in breast cancer that promotes a pro-metastatic tumor microenvironment. Oncogene. 2016;35(47):6053-64.
44. Fadok V A, Bratton D L, Konowal A, Freed P W, Westcott J Y, and Henson P M. Macrophages that have ingested apoptotic cells in vitro inhibit proinflammatory cytokine production through autocrine/paracrine mechanisms involving TGF-beta, PGE2, and PAF. The Journal of clinical investigation. 1998;101(4):890-8.
45. Lech M, and Anders H -J. Macrophages and fibrosis: How resident and infiltrating mononuclear phagocytes orchestrate all phases of tissue injury and repair. Biochimica et Biophysica Acta (BBA)—Molecular Basis of Disease. 2013;1832(7):989-97.
46. Minutti C M, Knipper J A, Allen J E, and Zaiss D M W. Tissue-specific contribution of macrophages to wound healing. Seminars in Cell & Developmental Biology. 2017;61:3-11.
47. Curtis C, Shah S P, Chin S -F, Turashvili G, Rueda O M, Dunning M J, et al. The genomic and transcriptomic architecture of 2,000 breast tumours reveals novel subgroups. Nature. 2012;486:346.
48. Kwong L S, Brown M H, Barclay A N, and Hatherley D. Signal-regulatory protein α from the NOD mouse binds human CD47 with an exceptionally high affinity—implications for engraftment of human cells. Immunology. 2014;143(1):61-7.
49. Hurvitz S A, Betting D J, Stern H M, Quinaux E, Stinson J, Seshagiri S, et al. Analysis of Fcgamma receptor IIIa and IIa polymorphisms: lack of correlation with outcome in trastuzumab-treated breast cancer patients. Clinical cancer research: an official journal of the American Association for Cancer Research. 2012;18(12):3478-86.
50. Barok M, Isola J, Pályi-Krekk Z, Nagy P, Juhász I, Vereb G, et al. Trastuzumab causes antibody-dependent cellular cytotoxicity-mediated growth inhibition of submacroscopic JIMT-1 breast cancer xenografts despite intrinsic drug resistance. Molecular Cancer Therapeutics. 2007;6(7):2065.
51. Ritter C A, Perez-Torres M, Rinehart C, Guix M, Dugger T, Engelman J A, et al. Human breast cancer cells selected for resistance to trastuzumab in vivo overexpress epidermal growth factor receptor and ErbB ligands and remain dependent on the ErbB receptor network. Clinical cancer research: an official journal of the American Association for Cancer Research. 2007;13(16):4909-19.
52. Yakes F M, Chinratanalab W, Ritter C A, King W, Seelig S, and Arteaga C L. Herceptin-induced inhibition of phosphatidylinositol-3 kinase and Akt Is required for antibody-mediated effects on p2′7, cyclin D1, and antitumor action. Cancer research. 2002;62(14):4132-41.
53. Grugan K D, McCabe F L, Kinder M, Greenplate A R, Harman B C, Ekert J E, et al. Tumor-Associated Macrophages Promote Invasion while Retaining Fc-Dependent Anti-Tumor Function. The Journal of Immunology. 2012;189(11):5457.
54. Gul N, and van Egmond M. Antibody-Dependent Phagocytosis of Tumor Cells by Macrophages: A Potent Effector Mechanism of Monoclonal Antibody Therapy of Cancer. Cancer Res. 2015;75(23):5008-13.
55. Brown J M, Recht L, and Strober S. The Promise of Targeting Macrophages in Cancer Therapy. Clinical cancer research: an official journal of the American Association for Cancer Research. 2017;23(13):3241-50.
56. Ruffell B, Affara N I, and Coussens L M. Differential macrophage programming in the tumor microenvironment. Trends Immunol. 2012;33(3):119-26.
57. Noy R, and Pollard J W. Tumor-associated macrophages: from mechanisms to therapy. Immunity. 2014;41(1):49-61.
58. Zhang Q W, Liu L, Gong C Y, Shi H S, Zeng Y H, Wang X Z, et al. Prognostic significance of tumor-associated macrophages in solid tumor: a meta-analysis of the literature. PLoS One. 2012;7(12):e50946.
59. Mantovani A, Marchesi F, Malesci A, Laghi L, and Allavena P. Tumour-associated macrophages as treatment targets in oncology. Nature reviews Clinical oncology. 2017;14(7):399-416.
60. Taylor C, Hershman D Fau-Shah N, Shah N Fau-Suciu-Foca N, Suciu-Foca N Fau-Petrylak DP, Petrylak Dp Fau-Taub R, Taub R Fau-Vandat L, et al. Augmented HER-2 specific immunity during treatment with trastuzumab and chemotherapy. (1078-0432 (Print)).
61. Abès R, Gélizé E, Fridman W H, and Teillaud J -L. Long-lasting antitumor protection by anti-CD20 antibody through cellular immune response. Blood. 2010;116(6):926.
62. Pfirschke C, Engblom C, Rickelt S, Cortez-Retamozo V, Garris C, Pucci F, et al. Immunogenic Chemotherapy Sensitizes Tumors to Checkpoint Blockade Therapy. Immunity. 2016;44(2):343-54.
63. Chao M P, Alizadeh A A, Tang C, Myklebust J H, Varghese B, Gill S, et al. Anti-CD47 antibody synergizes with rituximab to promote phagocytosis and eradicate non-Hodgkin lymphoma. Cell. 2010;142(5):699-713.
64. Kurebayashi J, Otsuki T, Tang C K, Kurosumi M, Yamamoto S, Tanaka K, et al. Isolation and characterization of a new human breast cancer cell line, KPL-4, expressing the Erb B family receptors and interleukin-6. Br J Cancer. 1999;79(5-6):707-17.
65. Shalem O, Sanjana N E, Hartenian E, Shi X, Scott D A, Mikkelsen T S, et al. Genome-Scale CRISPR-Cas9 Knockout Screening in Human Cells. Science. 2014;343(6166):84.
One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.
No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A method for treating a HER2/neu positive cancer in a subject in need thereof, the method comprising administering to the subject a therapeutically effective amount of a HER2 antibody comprising an IgG Fc portion capable of binding Fcγ-receptor (FCGR) and activating the antibody dependent cellular phagocytosis (ADCP) and a CD47 antagonist such that the cancer is treated in the subject.

2. The method according to claim 1, wherein the HER2 antibody is selected from the group consisting of trastuzumab, trastuzumab-dsk, MYL-1401O, ado-trastuzumab emtansine, pertuzumab and combinations thereof

3. The method according to claim 2, wherein the HER2 antibody is trastuzumab.

4. The method of claim 1, wherein the HER2 antibody has a high activating FcγR binding to inhibitory FcγR binding (A/I ratio) of greater than 1.

5. The method of claim 1, wherein the HER2 antibody has a human IgG1 Fc portion capable of activating the antibody dependent cellular phagocytosis (ADCP).

6. The method of claim 1, wherein the CD47 antagonist is selected from the group consisting of MIAP301, MIAP410, TTI-621, CV1, Hu5F9-G4, CC-90002, B6H12, 2D3 and combinations thereof.

7. The method of claim 6, wherein the CD47 antagonist is MIAP410.

8. The method of claim 1 in which the CD47 antagonist is administered prior to the HER2 antibody.

9. The method of claim 1, wherein the CD47 antagonist is administered concurrently with the HER2 antibody.

10. The method of claim 1, wherein the subject comprises a human.

11. method of claim 1, wherein the cancer comprises breast cancer.

12. The method of claim 1, wherein the subject also undergoes standard of care therapy.

13. The method of claim 1, wherein the subject is a subject that has a HER/neu+ positive cancer and the cancer expresses increased amounts of CD47 as compared to a control.

14. The method of claim 1, wherein the method further comprises: detecting a HER2/neu+ CD47+ cancer within a subject before administering the HER2 antibody and a CD47 antagonist.

15. A pharmaceutical composition comprising at least one HER2 antibody comprising an IgG Fc portion capable of binding Fcγ-receptor (FCGR) and activating the antibody dependent cellular phagocytosis (ADCP) and a CD47 antagonist for the treatment of HER2/neu positive cancer.

16. The pharmaceutical composition of claim 15, wherein the HER2 antibody is selected from the group consisting of trastuzumab, trastuzumab-dsk, MYL-1401O, lapatinib, neratinib, ado-trastuzumab emtansine, pertuzumab and combinations thereof.

17. The pharmaceutical composition of claim 15, wherein the HER2 antibody is trastuzumab.

18. The pharmaceutical composition of claim 15, wherein the HER2 antibody has a high activating FcγR binding to inhibitory FcγR binding (A/I ratio).

19. The pharmaceutical composition of claim 15, wherein the HER2 antibody has a human IgG1 Fc portion capable of activating the antibody dependent cellular phagocytosis (ADCP).

20. The pharmaceutical composition of claim 15, wherein the CD47 antagonist is selected from the group consisting of MIAP301, MIAP410, TTI-621, CV1, Hu5F9-G4, CC-90002, B6H12, 2D3 and combinations thereof.

21. (canceled)

22. (canceled)

23. A method comprising:
detecting in a tumor sample HER2/neu positive and CD47 positive tumor cells; and
administering to the subject a therapeutically effective amount of a HER2 antibody comprising an IgG Fc portion capable of binding Fcγ-receptor (FCGR) and activating the antibody dependent cellular phagocytosis (ADCP) and a CD47 antagonist if both HER2⁺ and CD47⁺ tumor cells are detected.
